(19) United States
(12) Patent Application Publication
(10) Pub. No.: US 2024/0115849 A1
(43) Pub. Date: Apr. 11, 2024

(54) CATHETER BLOOD PUMPS WITH COLLAPSIBLE PUMP HOUSING AND SENSOR SYSTEM

(71) Applicant: Shifamed Holdings, LLC, Campbell, CA (US)

(72) Inventors: Harshveer DHALIWAL, San Jose, CA (US); Michael CALOMENI, San Jose, CA (US); Brian D. BRANDT, Morgan Hill, CA (US); Ari RYAN, Sunnyvale, CA (US); Janine ROBINSON, Half Moon Bay, CA (US); Reza SHIRAZI, Campbell, CA (US)

(21) Appl. No.: 18/546,070

(22) PCT Filed: Feb. 10, 2022
(86) PCT No.: PCT/US2022/016009

§ 371 Date: Aug. 10, 2023

Related U.S. Application Data
(60) Provisional application No. 63/148,035, filed on Feb. 10, 2021.

(51) Int. Cl. A61M 60/13; A61M 60/531; A61M 60/804; A61M 60/816

(52) U.S. Cl. CPC A61M 60/13; A61M 60/531; A61M 60/804; A61M 60/816

(57) ABSTRACT

Catheter blood pumps that include a sensor system. The pumps may include a collapsible blood conduit that defines a blood lumen. The collapsible blood conduits include a collapsible scaffold adapted to provide radial support to the blood conduit. The pumps may also include one or more impellers. One or more sensors may be arranged with respect to inflow and outflow regions of the blood lumen, the scaffold and/or the impellers.

### CROSS REFERENCE TO RELATED APPLICATIONS

This application claims the benefit of U.S. Application No. 63/148,035 filed Feb. 10, 2021, entitled “Catheter Blood Pumps With Collapsible Pump Housing and Sensor System,” the entirety of which is incorporated herein for all purposes. This application may also be related to International Application No. PCT/US2020/052877, filed Sep. 25, 2020, and entitled “Catheter Blood Pumps And Collapsible Pump Housings”, the entirety of which is incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally-invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously-inserted ventricular support device.

There is a need to provide additional improvements to the field of ventricular support devices and similar blood pumps for treating compromised cardiac blood flow.

### SUMMARY OF THE DISCLOSURE

The disclosure is related to intravascular blood pumps and their methods of use and manufacture.

One aspect of the disclosure is a catheter blood pump, comprising: a blood conduit defining a lumen and having a first end and a second end, wherein the first end is at an inflow region of the blood conduit and the second end is at an outflow region of the blood conduit; an impeller positioned at least partially within the lumen of the blood conduit and configured to move blood from the inflow region toward the outflow region; a plurality of struts extending from the first end or the second end of the blood conduit; and a pressure transducer positioned between two adjacent struts in the inflow region or the outflow region of the blood conduit.

In this aspect, the pressure sensor transducer has a pressure sensitive face may be facing outward relative to a long axis of catheter blood pump.

In this aspect, the pressure transducer may have a pressure sensitive face oriented non-orthogonally relative to a long axis of the catheter pump.

In this aspect, the catheter blood pump may further comprise a pressure sensor housing in which the pressure transducer is securely disposed.

In this aspect, the pressure sensor housing may include a proximal opening sized and configured to receive a pressure sensor wire coupled to the pressure transducer.

In this aspect, the catheter blood pump may further comprise a pressure sensor wire extending proximally from the pressure transducer.

In this aspect, the outflow region may be at a proximal end of the blood conduit, wherein the plurality of struts extend in a proximal direction relative to the blood conduit.

In this aspect, the proximal ends of the plurality of struts may be coupled to a hub of the catheter blood pump.

In this aspect, the inflow region may be at a distal end of the blood conduit, wherein the plurality of struts extend in a distal direction relative to the blood conduit.

In this aspect, distal ends of the plurality of struts may be coupled to a central hub of the catheter blood pump.

In this aspect, the catheter blood pump may include a proximal pressure transducer in the outflow region and a distal pressure transducer in the inflow region.

In this aspect, the catheter blood pump may further comprise a first wire electrically coupled to the proximal pressure transducer and a second wire electrically coupled to the distal pressure transducer.

In this aspect, the first wire and the second wire may extend together at a junction region, wherein the junction region is proximally located with respect to the blood conduit.

In this aspect, the junction region may be proximally located with respect to the proximal pressure sensor.

In this aspect, the plurality of struts may be expandable and collapsible.

In this aspect, the blood conduit may be expandable and collapsible.

In this aspect, the impeller may be expandable and collapsible.

In this aspect, the catheter blood pump may further comprise a second impeller positioned at least partially within the blood conduit.

In this aspect, the inflow region may include a distal end of the blood conduit and the outflow region includes a proximal end of the blood conduit.

In this aspect, the pressure transducer may be positioned circumferentially between the two adjacent struts of the plurality of struts, optionally circumferentially between feet (or axially extending regions) of the two adjacent struts.

One aspect of the disclosure is a catheter blood pump coupled to a distal end of a catheter shaft, the catheter blood pump comprising: a blood conduit having an impeller at least partially positioned therein, the blood conduit having a first end and a second end; a proximal pressure transducer in an outflow region of the catheter blood pump; a distal pressure transducer in an inflow region of the catheter blood pump; and a first wire in communication with the proximal pressure transducer and a second wire in communication with the distal pressure transducer, the first and second wires extending together along a length of the catheter shaft, wherein the first wire follows a first path along a proximal central hub of the catheter blood pump to the proximal pressure transducer, and wherein the second wire follows a second path along the blood conduit to the distal pressure transducer.

In this aspect, the catheter blood pump may further comprise a plurality of proximal struts extending from a proximal end of the blood conduit, wherein the proximal pressure transducer is positioned between two of the plurality of proximal struts, optionally circumferentially between feet of the plurality of proximal struts.

In this aspect, the plurality of proximal struts may be expandable and collapsible.

In this aspect, the second path of the second wire may be along one of the plurality of proximal struts (optionally along an outer surface of the proximal strut).

In this aspect, the catheter blood pump may further comprise a plurality of distal struts extending from a distal end of the blood conduit, wherein the distal pressure transducer is disposed between two of the plurality of distal struts, optionally circumferentially between feet of the plurality of distal struts.

In this aspect, the second wire may follow a path along one of the plurality of distal struts (optionally along an outer surface of the distal strut).

In this aspect, the proximal pressure transducer and the distal pressure transducer may each have a pressure sensitive face facing outward relative to a long axis of the catheter blood pump.

In this aspect, the proximal pressure transducer and the distal pressure transducer may each have a pressure sensitive face oriented non-orthogonally relative to a long axis of the catheter pump.

In this aspect, a portion of the second wire may extend distally from the distal pressure transducer.

In this aspect, a portion of the second wire may have a curved configuration such that a portion of the second wire extends circumferentially around at least a portion of a distal hub to which the distal pressure transducer is secured.

In this aspect, the second wire distal hub may include a recessed channel in which the second wire is disposed and that secures the second wire in the curved configuration.

One aspect of the disclosure is a catheter blood pump, comprising: blood conduit having one or more impellers at least partially disposed therein, the one or more impellers configured to move blood from an inflow region at the distal end of the blood conduit toward an outflow region at the proximal end of the blood conduit; a plurality of struts extending from the distal end of the blood conduit and coupled to a distal central hub; a pressure transducer at the inflow region, the pressure transducer disposed on the distal central hub and between two of the plurality of struts; and a wire extending along one of the plurality of struts and operationally coupled to the pressure transducer.

In this aspect, the pressure transducer may be disposed between a first strut and a second strut, wherein the pressure transducer wire follows a path along a third strut different than the first strut and the second strut.

In this aspect, the pressure transducer may be disposed between a first strut and a second strut, wherein the wire follows a path along the first strut or the second distal strut.

In this aspect, the wire may have a curved configuration in which the wire traverses circumferentially around at least a portion of the distal central hub.

In this aspect, the distal hub may include a recessed channel that accommodates a portion of the wire in the curved configuration.

In this aspect, the pressure transducer may be a distal pressure transducer, wherein the catheter blood pump further comprises a proximal pressure transducer at the outflow region.

In this aspect, the plurality of struts may be distal struts, wherein the catheter blood pump further comprises a plurality of proximal struts extending from the proximal end of the blood conduit.

In this aspect, the struts may be expandable and collapsible.

In this aspect, the blood conduit may be expandable and collapsible.

One aspect of the disclosure is a catheter blood pump, comprising: blood conduit having one or more impellers at least partially disposed therein, the one or more impeller configured to move blood from an inflow region at the distal end of the blood conduit toward an outflow region at the proximal end of the blood conduit; a proximal pressure transducer at the outflow region; a distal pressure transducer at the inflow region; and a first wire in communication with the proximal transducer and a second wire in communication with the distal pressure transducer, the first wire extending proximally from the proximal transducer, the second wire extending distally from the distal transducer.

In this aspect, the distal pressure sensor may be distally located with respect to the blood conduit.

In this aspect, the catheter blood pump may further comprise proximal struts extending from the proximal end of the blood conduit and distal struts extending from the distal end of the blood conduit.

In this aspect, the distal pressure sensor may be coupled to a distal central hub to which the distal struts are secured, the distal pressure sensor disposed between two of the distal struts.

In this aspect, a portion of the second wire may extend through a channel of the distal central hub.

In this aspect, the second wire may extend distally from the distal sensor and extends proximally along the blood conduit.

In this aspect, a portion of the second wire may extend circumferentially around at least a portion of a catheter shaft that is coupled to the catheter blood pump.

One aspect of the disclosure is a catheter blood pump, comprising: a blood conduit defining a lumen and having a first end and a second end, wherein the first end is at an inflow region and the second end is at an outflow region; one or more impellers at least partially disposed within the lumen of the blood conduit and configured to move blood from the inflow region toward the outflow region; a distal central hub coupled to the blood conduit, the distal central hub including: a pressure transducer at the inflow region; a channel configured to accommodate a wire in communication with the pressure transducer, wherein the channel has a curved configuration to guide the wire from the pressure transducer and circumferentially around at least a portion the distal central hub.

In this aspect, the catheter blood pump may further comprise struts extending from the second end of the blood conduit to the distal central hub.

In this aspect, the pressure transducer may be coupled to the distal central hub between two of the struts.

In this aspect, the channel may guide the wire toward one of the struts.

In this aspect, the wire may follow a path along the one of the struts.

In this aspect, the channel may be distally located with respect to the struts.

In this aspect, at least one of the struts may include a t-shaped end secured within a groove in the distal central hub.

In this aspect, the catheter blood pump may further comprise struts extending from the first end of the blood conduit to a proximal central hub.

In this aspect, the catheter blood pump may further comprise a second pressure transducer coupled to the proximal central hub between two of the struts.

In this aspect, at least one of the struts may include a t-shaped end secured within a groove in the proximal central hub.

One aspect of the disclosure is a catheter blood pump, comprising: blood conduit defining an inner lumen, the blood conduit including one or more impellers at least partially disposed with the inner lumen, the one or more impellers configured to move blood from an inflow region at the distal end of the blood conduit toward an outflow region at the proximal end of the blood conduit; a pressure transducer at the inflow region of the blood conduit; and a wire in communication with the pressure transducer, wherein at least a portion of the wire extends distally from the pressure transducer.

In this aspect, at least a portion of the wire may extend along a central hub of the catheter blood pump.

In this aspect, the catheter blood pump may further comprise struts extending from the blood conduit to a central hub of the catheter blood pump.

In this aspect, the struts may extend from the distal end of the blood conduit at the inflow region.

In this aspect, the pressure transducer may be coupled to a distal central hub and between two of the struts.

In this aspect, the distal central hub may cover a bearing assembly.

In this aspect, the struts may extend from the proximal end of the blood conduit at the outflow region.

In this aspect, the pressure transducer may be coupled to a proximal central hub and between two of the struts.

In this aspect, the proximal central hub may cover a bearing assembly.

One aspect of the disclosure is a catheter blood pump, comprising: a blood conduit defining a lumen and having a first end and a second end, wherein the first end is at an outflow region of the blood conduit and the second end is at an inflow region of the blood conduit; an impeller positioned at least partially within the lumen of the blood conduit and configured to move blood from the inflow region toward the outflow region; and a proximal central hub coupled to the blood conduit, the proximal central hub including a first sensor housing at the outflow region of the blood conduit, the first sensor housing having a first pressure transducer secured therein and a first aperture that guides a first wire from the first pressure transducer in a proximal direction along the proximal central hub; and a distal central hub coupled to the blood conduit, the distal central hub including a second sensor housing at the inflow region of the blood conduit, the second sensor housing having a second pressure transducer secured therein and a second aperture that guides a second wire from the second pressure transducer in a distal direction along the distal central hub.

In this aspect, the catheter blood pump may be coupled to a distal end of a catheter shaft, wherein the first and second wires extend along an outer surface of the catheter shaft.

In this aspect, at least a portion of the first and second wires may wind in a helical configuration around the catheter shaft.

In this aspect, a pitch of the first and second wired winding around the catheter shaft may vary axially along the catheter shaft.

In this aspect, at least another portion of the first and second wires may follow a path that is parallel to the catheter shaft (non-winding).

In this aspect, a portion of the second wire may traverse along the blood conduit between the outflow region and the inflow region.

In this aspect, a portion of the second wire may extend circumferentially around at least a portion of the distal hub.

In this aspect, the distal hub may include a recessed channel that accommodates a portion of the second wire in a curved configuration.

One aspect of the disclosure is a catheter blood pump, comprising: a catheter and an expandable blood pump secured thereto, the expandable blood pump including an expandable blood conduit, a plurality of struts (e.g., proximal struts or distal struts) extending axially away from the blood conduit, and an impeller at least partially disposed in the blood conduit; and at least one of a pressure sensor (e.g., any of the pressure sensors/transducers herein) or pressure sensor housing disposed circumferentially between feet of the first and second struts of the plurality of struts and wherein the at least one of the pressure sensor or pressure sensor housing axially overlaps with the feet of the first and second struts.

This aspect may further comprise a collapsible impeller housing comprising the collapsible blood conduit and the plurality of struts, wherein a pressure sensitive surface of the pressure sensor may be disposed at least partially axially outside of the collapsible impeller housing.

These and other aspects are described herein.

### BRIEF DESCRIPTION OF THE DRAWINGS

FIG. 1 is a side view of an exemplary expandable pump portion that includes an expandable impeller housing that includes a scaffold and blood conduit, and a plurality of impellers.

FIG. 2 is a side view of an exemplary expandable pump portion that includes an expandable impeller housing, a blood conduit, a plurality of impellers, and a plurality of expandable scaffolds sections or support members.

FIGS. 3A, 3B, 3C and 3D illustrate an exemplary expandable pump portion that includes a blood conduit, a plurality of impellers, and a plurality of expandable scaffold sections or support members.

FIG. 4 illustrates an exemplary target location of an expandable pump portion, the pump portion including a blood conduit, a plurality of expandable scaffold sections or support members, and a plurality of impellers.

FIG. 5 illustrates an exemplary pump portion including an expandable impeller housing, a blood conduit, and a plurality of impellers.

FIG. 6A illustrates at least a portion of an exemplary catheter blood pump that includes a pump portion, wherein at least two different impellers can be rotated at different speeds.

FIG. 6B illustrates at least a portion of an exemplary catheter blood pump that includes a pump portion, where at least two different impellers can be rotated at different speeds.

FIG. 6C illustrates at least a portion of an exemplary catheter blood pump that includes a pump portion with at least two impellers having different pitches.

FIG. 7 illustrates a portion of an exemplary catheter blood pump that includes a pump portion.

FIG. 8 illustrates an exemplary expandable pump portion including a plurality of expandable impellers, including one or more bends formed therein between adjacent impellers.

FIG. 9 illustrates an exemplary expandable pump portion comprising a plurality of impellers and a blood conduit.

FIG. 10 illustrates an exemplary scaffold design and exemplary struts.

FIG. 11 illustrate an exemplary scaffold design and exemplary struts.

FIGS. 12A-12F illustrate an exemplary sequence of steps that may be performed to deploy an exemplary pump portion of a catheter blood pump.

FIGS. 13A and 13B illustrate exemplary portions of an expandable pump portion.

FIG. 13C illustrates a scaffold from FIGS. 13A and 13B shown in a flattened and non-expanded configuration, as well as optional distal and proximal struts extending axially therefrom.

FIG. 14A illustrates an exemplary expanded scaffold that may be part of any of the expandable pump portions herein.

FIG. 14B illustrates the scaffold and struts from FIG. 14A in a flattened and non-expanded configuration.

FIG. 15A illustrates an exemplary expanded scaffold that may be part of any of the expandable pump portions herein.

FIG. 15B illustrates the scaffold and struts from FIG. 15A in a flattened and non-expanded configuration.

FIG. 16 illustrates an exemplary scaffold and optionally coupled struts in a flattened and non-expanded configuration.

FIG. 17 illustrates an exemplary scaffold and optionally coupled struts in a flattened and non-expanded configuration.

FIG. 18A illustrates an exemplary scaffold in a flattened and non-expanded configuration.

FIG. 18B illustrates the scaffold from FIG. 18A in an expanded configuration.

FIG. 19A illustrates an exemplary scaffold in a flattened and non-expanded configuration.

FIG. 19B illustrates the scaffold from FIG. 19A in an expanded configuration.

FIG. 20A illustrates an exemplary scaffold in a flattened and non-expanded configuration.

FIG. 20B illustrates the scaffold from FIG. 20A in an expanded configuration.

FIG. 21A illustrates an exemplary scaffold in a flattened and non-expanded configuration.

FIG. 21B illustrates the scaffold from FIG. 21A in an expanded configuration.

FIG. 22A illustrates an exemplary scaffold in a flattened and non-expanded configuration.

FIG. 22B illustrates the scaffold from FIG. 22A in an expanded configuration.

FIG. 23A illustrates an exemplary scaffold in a flattened and non-expanded configuration.

FIG. 23B illustrates the scaffold from FIG. 23A in an expanded configuration.

FIG. 24A illustrates an exemplary scaffold in a flattened and non-expanded configuration.

FIG. 24B illustrates the scaffold from FIG. 24A in an expanded configuration.

FIG. 25A illustrates an exemplary scaffold in a flattened and non-expanded configuration.

FIG. 25B illustrates the scaffold from FIG. 25A in a flattened expanded configuration.

FIG. 26A illustrates an exemplary scaffold in a flattened and non-expanded configuration.

FIG. 26B highlights an exemplary section of the scaffold shown in FIG. 26A.

FIG. 27A illustrates an exemplary scaffold in a flattened and non-collapsed configuration.

FIG. 27B illustrates the scaffold from FIG. 27A in a non-collapsed configuration.

FIG. 28 is a side view of an exemplary pump portion that includes a sensor wire.

FIG. 29 is a cross sectional view of an exemplary expandable impeller housing that includes a sensor connector fixed to the expandable impeller housing.

FIG. 30 is a cross sectional view of an exemplary expandable impeller housing that includes a sensor connector disposed in a sensor wire lumen.

FIG. 31 is a cross sectional view of an exemplary expandable impeller housing that includes a sensor connector disposed in a sensor wire lumen.

FIG. 32 is a cross sectional view of an exemplary expandable impeller housing that includes a sensor connector disposed in a sensor wire lumen.

FIG. 33 is a side view of an exemplary pump portion that includes a sensor connector carried by and outside of an expandable impeller housing, the pump portion including a sensor coupled to the sensor wire.

FIG. 34A illustrates a region of an exemplary pump portion that includes a pressure sensor carrier or housing with a pressure sensor secured relative thereto.

FIG. 34B illustrates an exemplary sensor carrier or housing with an exemplary sensor secured relative thereto.

FIG. 35A illustrates a side view of an exemplary catheter blood pump with a sensor arrangement.

FIG. 35B illustrates a closeup view of the catheter blood pump of FIG. 35A, showing a proximal sensor housing.

FIG. 35C illustrates a closeup view of an exemplary pressure sensor, which may be used in the catheter blood pump of FIG. 35A.

FIG. 35D illustrates a closeup view of the proximal region of the catheter blood pump of FIG. 35A, showing a sensor connector arrangement.

FIG. 35E illustrates a closeup view of a distal region of the catheter blood pump of FIG. 35A, showing a distal sensor housing.

FIG. 35F illustrates a closeup view of the distal region of the pump portion of the catheter blood pump of FIG. 35A, showing a sensor connector arrangement.

FIGS. 36A and 36B illustrate partial flattened views of an exemplary scaffold that may be used as part of a blood conduit: FIG. 36A shows a proximal end of the scaffold having five struts; and FIG. 36B shows a distal end of the scaffold having five struts.

FIGS. 37A-37C illustrate another embodiment of a blood pump with a sensor on a distal portion.

FIG. 38 is another view of a blood pump with a sensor on a proximal portion.

### DETAILED DESCRIPTION

The present disclosure is related to medical devices, systems, and methods of use and manufacture. Medical devices herein may include a distal pump portion (which may also be referred to herein as a working portion) adapted to be disposed within a physiologic vessel, wherein the distal pump portion includes one or more components that act upon fluid. For example, pump portions herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

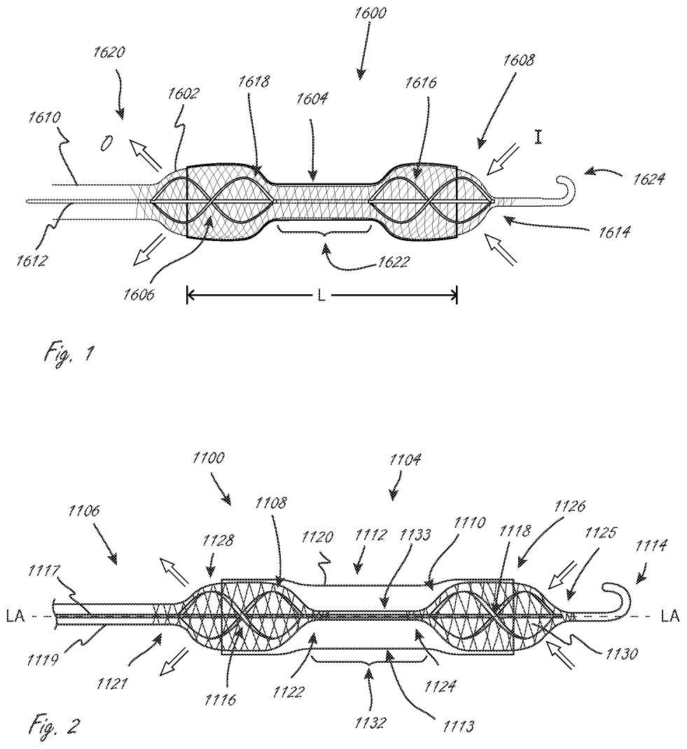

FIG. 1 is a side view illustrating a distal portion of an exemplary catheter blood pump, including pump portion 1600, wherein pump portion 1600 includes proximal impeller 1606 and distal impeller 1616, both of which are in operable communication with drive cable 1612. Although the embodiment of FIG. 1 shows two impellers, it should be understood that other embodiments (and any embodiment of a blood pump herein) can include only a single impeller, such as only a proximal impeller or only a distal impeller. Pump portion 1600 is in an expanded configuration in FIG. 1, but is adapted to be collapsed to a delivery configuration so that it can be delivered with a lower profile. The impellers can be attached to drive mechanism 1612 (e.g., a drive cable). Drive mechanism 1612 is in operable communication with an external motor, not shown, and extends through elongate shaft 1610. The phrases “pump portion” and “working portion” (or derivatives thereof) may be used herein interchangeably unless indicated to the contrary. For example without limitation, “pump portion” 1600 can also be referred to herein as a “working portion.”

Pump portion 1600 also includes expandable member or expandable scaffold 1602, which in this embodiment has a proximal end 1620 that extends further proximally than a proximal end of proximal impeller 1606, and a distal end 1608 that extends further distally than a distal end 1614 of distal impeller 1616. Expandable members may also be referred to herein as expandable scaffolds or scaffold sections. Expandable scaffold 1602 is disposed radially outside of the impellers along the axial length of the impellers. Expandable scaffold 1602 can be constructed in a manner and made from materials similar to many types of expandable structures that are known in the medical arts to be able to collapse and expand, examples of which are provided herein. Examples of suitable materials include, but are not limited to, polyurethane, polyurethane elastomers, metallic alloys, etc.

Pump portion 1600 also includes blood conduit 1604, which is coupled to and supported by expandable member 1602, has a length L, and extends axially between the impellers. Conduit 1604 creates and provides a fluid lumen between the two impellers. When in use, fluid moves through the lumen defined by conduit 1604. The conduits herein may be non-permeable, or they may be semipermeable, or even porous as long as they still define a lumen. The conduits herein are also flexible, unless otherwise indicated. The conduits herein extend completely around (i.e., 360 degrees) at least a portion of the pump portion. In pump portion 1600, the conduit extends completely around expandable member 1602, but does not extend all the way to the proximal end 1602 or distal end 1608 of expandable member 1602. The structure of the expandable member creates at least one inlet aperture to allow for inflow “I,” and at least one outflow aperture to allow for outflow “O.” Conduit 1604 improves impeller pumping dynamics, compared to pump portions without a conduit. As described herein, expandable members or scaffolds may also be considered to be a part of the blood conduit generally, which together define a blood lumen. In these instances the scaffold and material supported by the scaffold may be referred to herein as an expandable impeller housing or housing.

Expandable member 1602 may have a variety of constructions, and made from a variety of materials. For example, expandable member 1602 may be formed similar to expandable stents or stent-like devices, or any other example provided herein. For example without limitation, expandable member 1602 could have an open-braided construction, such as a 24-end braid, although more or fewer braid wires could be used. Exemplary materials for the expandable member as well as the struts herein include nitinol, cobalt alloys, and polymers, although other materials could be used. Expandable member 1602 has an expanded configuration, as shown, in which the outer dimension (measured orthogonally relative a longitudinal axis of the working portion) of the expandable member is greater in at least a region where it is disposed radially outside of the impellers than in a central region 1622 of the expandable member that extends axially between the impeller. Drive mechanism 1612 is co-axial with the longitudinal axis in this embodiment. In use, the central region can be placed across a valve, such as an aortic valve. In some embodiments, expandable member 1602 is adapted and constructed to expand to an outermost dimension of 12-24F (4.0-8.0 mm) where the impellers are axially within the expandable member, and to an outermost dimension of 10-20F (3.3-6.7 mm) in central region 1622 between the impellers. The smaller central region outer dimension can reduce forces acting on the valve, which can reduce or minimize damage to the valve. The larger dimensions of the expandable member in the regions of the impellers can help stabilize the working portion axially when in use. Expandable member 1602 has a general dumbbell configuration. Expandable member 1602 has an outer configuration that tapers as it transitions from the impeller regions to central region 1622, and again tapers at the distal and proximal ends of expandable member 1602.

Expandable member 1602 has a proximal end 1620 that is coupled to shaft 1610, and a distal end 1608 that is coupled to distal tip 1624. The impellers and drive mechanism 1612 rotate within the expandable member and conduit assembly. Drive mechanism 1612 is axially stabilized with respect to distal tip 1624, but is free to rotate with respect to tip 1624.

In some embodiments, expandable member 1602 can be collapsed by pulling tension from end-to-end on the expandable member. This may include linear motion (such as, for example without limitation, 5-20 mm of travel) to axially extend expandable member 1602 to a collapsed configuration with collapsed outer dimension(s). Expandable member 1602 can also be collapsed by pushing an outer shaft such as a sheath over the expandable member/conduit assembly, causing the expandable member and conduit to collapse towards their collapsed delivery configuration.

Impellers 1606 and 1616 are also adapted and constructed such that one or more blades will stretch or radially compress to a reduced outermost dimension (measured orthogonally to the longitudinal axis of the working portion). For example without limitation, any of the impellers herein can include one or more blades made from a plastic formulation with spring characteristics, such as any of the impellers described in U.S. Pat. No. 7,393,181, the disclosure of which is incorporated by reference herein for all purposes and can be incorporated into embodiments herein unless this disclosure indicates to the contrary. Alternatively, for example, one or more collapsible impellers can comprise a superelastic wire frame, with polymer or other material that acts as a webbing across the wire frame, such as those described in U.S. Pat. No. 6,533,716, the disclosure of which is incorporated by reference herein for all purposes.

The inflow and/or outflow configurations of working portion 1600 can be mostly axial in nature.

Exemplary sheathing and unsheathing techniques and concepts to collapse and expand medical devices are known, such as, for example, those described and shown in U.S. Pat. No. 7,841,976 or U.S. Pat. No. 8,052,749, the disclosures of which are incorporated by reference herein.

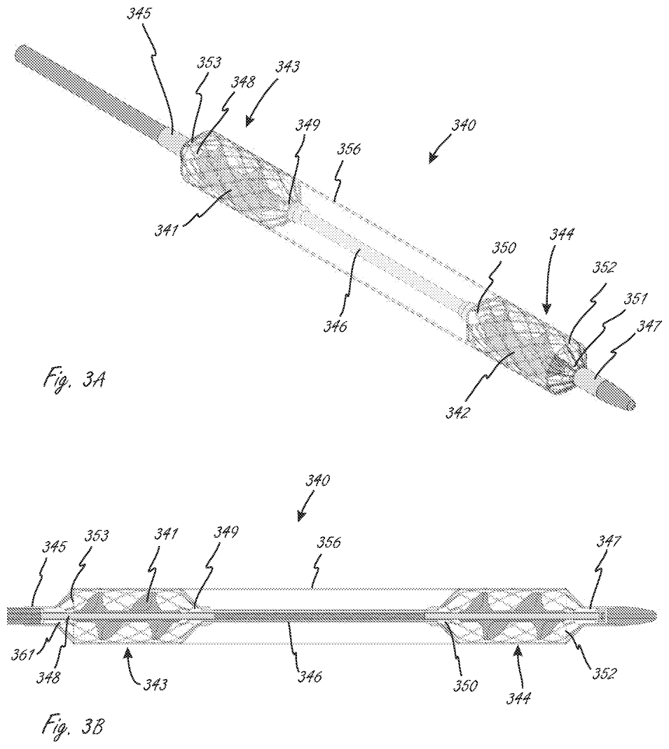

FIG. 2 is a side view illustrating a deployed configuration (shown extracorporally) of a distal portion of an exemplary embodiment of a catheter blood pump. Exemplary blood pump 1100 includes working portion 1104 (which as set forth herein may also be referred to herein as a pump portion) and an elongate portion 1106 extending from working portion 1104. Elongate portion 1106 can extend to a more proximal region of the system, not shown for clarity, and that can include, for example, a motor. Working portion 1104 includes first expandable scaffold or member 1108 and second expandable scaffold or member 1110, axially spaced apart along a longitudinal axis LA of working portion 1104. First scaffold 1108 and second scaffold 1110 (and any other separate scaffolds herein) may also be referenced as part of a common scaffold and referred to herein as scaffold sections. Spaced axially in this context refers to the entire first expandable member being axially spaced from the entire second expandable member along a longitudinal axis LA of working portion 1104. A first end 1122 of first expandable member 1108 is axially spaced from a first end 1124 of second expandable member 1110.

First and second expandable members 1108 and 1110 generally each include a plurality of elongate segments disposed relative to one another to define a plurality of apertures 1130, only one of which is labeled in the second expandable member 1110. The expandable members can have a wide variety of configurations and can be constructed in a wide variety of ways, such as any of the configurations or constructions in, for example without limitation, U.S. Pat. No. 7,841,976, or the tube in 6,533,716, which is described as a self-expanding metal endoprosthetic material. For example, without limitation, one or both of the expandable members can have a braided construction or can be at least partially formed by laser cutting a tubular element.

Working portion 1104 also includes blood conduit 1112 that is coupled to first expandable member 1108 and to second expandable member 1110, and extends axially in between first expandable member 1108 and second expandable member 1110 in the deployed configuration. A central region 1113 of conduit 1112 spans an axial distance 1132 where the working portion is void of first and second expandable members 1108 and 1110. Central region 1113 can be considered to be axially in between the expandable members. Distal end 1126 of conduit 1112 does not extend as far distally as a distal end 1125 of second expandable member 1110, and proximal end of conduit 1128 does not extend as far proximally as proximal end 1121 of first expandable member 1108.

When the disclosure herein refers to a blood conduit being coupled to an expandable scaffold or member, the term coupled in this context does not require that the conduit be directly attached to the expandable member so that conduit physically contacts the expandable member. Even if not directly attached, however, the term coupled in this context refers to the conduit and the expandable member being joined together such that as the expandable member expands or collapses, the conduit also begins to transition to a different configuration and/or size. Coupled in this context therefore refers to conduits that will move when the expandable member to which it is coupled transitions between expanded and collapsed configurations.

Any of the blood conduits herein can be deformable to some extent. For example, conduit 1112 includes elongate member 1120 that can be made of one or more materials that allow the central region 1113 of conduit to deform to some extent radially inward (towards LA) in response to, for example and when in use, forces from valve tissue (e.g., leaflets) or a replacement valve as working portion 1104 is deployed towards the configuration shown in FIG. 2. The conduit may be stretched tightly between the expandable members in some embodiments. The conduit may alternatively be designed with a looseness that causes a greater degree of compliance. This can be desirable when the working portion is disposed across fragile structures such as an aortic valve, which may allow the valve to compress the conduit in a way that minimizes point stresses in the valve. In some embodiments, the conduit may include a membrane attached to the proximal and distal expandable members. Exemplary materials that can be used for any conduits herein include, without limitations, polyurethane rubber, silicone rubber, acrylic rubber, expanded polytetrafluoroethylene, polyethylene, polyethylene terephthalate, including any combination thereof.

Any of the conduits herein can have a thickness of, for example, 0.5-20 thousandths of an inch (thou), such as 1-15 thou, or 1.5 to 15 thou, 1.5 to 10 thou, or 2 to 10 thou.

Any of the blood conduits herein, or at least a portion of the conduit, can be impermeable to blood. In FIG. 2, working portion 1104 includes a lumen that extends from distal end 1126 of conduit 1112 and extends to proximal end 1128 of conduit 1112. The lumen is defined by conduit 1112 in central region 1113, but can be thought of being defined by both the conduit and portions of the expandable members in regions axially adjacent to central region 1113. In this embodiment, however, it is the conduit material that causes the lumen to exist and prevents blood from passing through the conduit.

Any of the conduits herein that are secured to one or more expandable members can be, unless indicated to the contrary, secured so that the conduit is disposed radially outside of one or more expandable members, radially inside of one or more expandable members, or both, and the expandable member can be impregnated with the conduit material.

The proximal and distal expandable scaffolds or members help maintain the blood conduit in an open configuration to create the lumen, while each also creates a working environment for an impeller, described below. Each of the expandable scaffolds, when in the deployed configuration, is maintained in a spaced relationship relative to a respective impeller, which allows the impeller to rotate within the expandable member without contacting the expandable member. Working portion 1104 includes first impeller 1116 and second impeller 1118, with first impeller 1116 disposed radially within first expandable member 1108 and second impeller 1118 disposed radially within second expandable member 1110. In this embodiment, the two impellers even though they are distinct and separate impellers, are in operable communication with a common drive mechanism (e.g., drive cable 1117), such that when the drive mechanism is activated the two impellers rotate together. In this deployed configuration, impellers 1116 and 1118 are axially spaced apart along longitudinal axis LA, just as are the expandable members 1108 and 1110 are axially spaced apart. As described above, however, any of the devices and methods described herein can include only a single impeller, such as a proximal impeller or a distal impeller.

Impellers 1116 and 1118 are also axially within the ends of expandable members 1108 and 1110, respectively (in addition to being radially within expandable members 1108 and 1110). The impellers herein can be considered to be axially within an expandable member even if the expandable member includes struts extending from a central region of the expandable member towards a longitudinal axis of the working portion (e.g., tapering struts in a side view). In FIG. 2, second expandable member 1110 extends from first end 1124 (proximal end) to second end 1125 (distal end). As described above, any embodiments of blood pumps can include only a single impeller instead of two impellers, such as only a proximal impeller or only a distal impeller.

In FIG. 2, a distal portion of impeller 1118 extends distally beyond distal end 1126 of conduit 1112, and a proximal portion of impeller 1116 extends proximally beyond proximal end 1128 of conduit 1112. In this figure, portions of each impeller are axially within the conduit in this deployed configuration.

In the exemplary embodiment shown in FIG. 2, impellers 1116 and 1118 are in operable communication with a common drive mechanism 1117, and in this embodiment, the impellers are each coupled to drive mechanism 1117, which extends through shaft 1119 and working portion 1104. Drive mechanism 1117 can be, for example, an elongate drive cable, which when rotated causes the impellers to rotate. In this example, as shown, drive mechanism 1117 extends to and is axially fixed relative to distal tip 1114, although it is adapted to rotate relative to distal tip 1114 when actuated. Thus, in this embodiment, the impellers and drive mechanism 1117 rotate together when the drive mechanism is rotated. Any number of known mechanisms can be used to rotate drive mechanism, such as with a motor (e.g., an external motor).

The expandable members and the conduit are not in rotational operable communication with the impellers and the drive mechanism. In this embodiment, proximal end 1121 of proximal expandable member 1108 is coupled to shaft 1119, which may be a shaft of elongate portion 1106 (e.g., an outer catheter shaft). Distal end 1122 of proximal expandable member 1108 is coupled to central tubular member 1133, through which drive mechanism 1117 extends. Central tubular member 1133 extends distally from proximal expandable member 1108 within conduit 1112 and is also coupled to proximal end 1124 of distal expandable member 1110. Drive mechanism 1117 thus rotates within and relative to central tubular member 1133. Central tubular member 1133 extends axially from proximal expandable member 1108 to distal expandable member 1110. Distal end 1125 of distal expandable member 1110 is coupled to distal tip 1114, as shown. Drive mechanism 1117 is adapted to rotate relative to tip 1114, but is axially fixed relative to tip 1114.

Working portion 1104 is adapted and configured to be collapsed to a smaller profile than its deployed configuration (which is shown in FIG. 2). This allows it to be delivered using a lower profile delivery device (smaller French size) than would be required if none of working portion 1104 was collapsible. Even if not specifically stated herein, any of the expandable members and impellers may be adapted and configured to be collapsible to some extent to a smaller delivery configuration.

The working portions herein can be collapsed to a collapsed delivery configuration using conventional techniques, such as with an outer sheath that is movable relative to the working portion (e.g., by axially moving one or both of the sheath and working portion). For example without limitation, any of the systems, devices, or methods shown in the following references may be used to facilitate the collapse of a working portions herein: U.S. Pat. No. 7,841,976 or U.S. Pat. No. 8,052,749, the disclosures of which are incorporated by reference herein for all purposes.

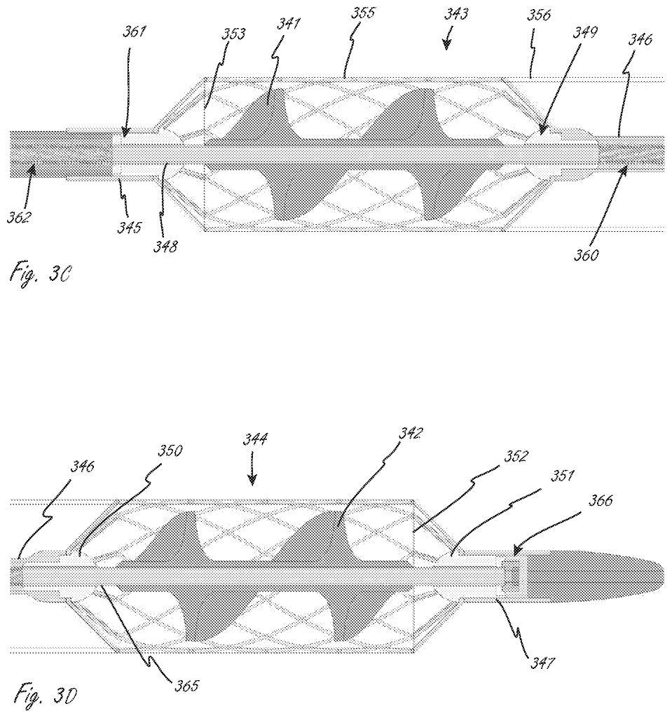

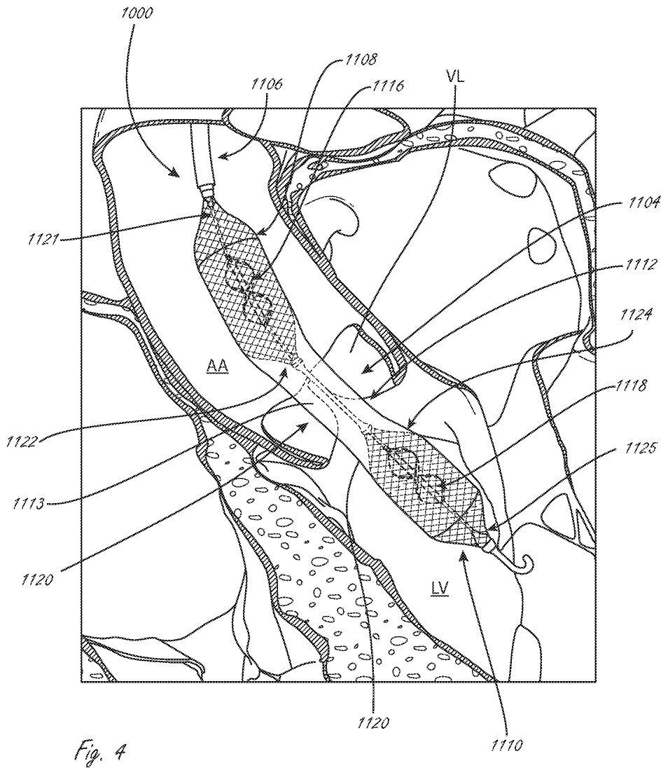

FIGS. 3A-3D show an exemplary pump portion that is similar in some ways to the pump portion shown in FIG. 2. Pump portion 340 is similar to pump portion 1104 in that in includes two expandable members axially spaced from one another when the pump portion is expanded, and a conduit extending between the two expandable members. FIG. 3A is a perspective view, FIG. 3B is a side sectional view, and FIGS. 3C and 3D are close-up side sectional views of sections of the view in FIG. 3B.

Pump portion 340 includes proximal impeller 341 and distal impeller 342, which are coupled to and in operational communication with a drive cable, which defines therein a lumen. In some embodiments, however, the pump portion includes only a single impeller, such as only a proximal impeller or only a distal impeller. The lumen can be sized to accommodate a guidewire, which can be used for delivery of the working portion to the desired location. The drive cable, in this embodiment, includes first section 362 (e.g., wound material), second section 348 (e.g., tubular member) to which proximal impeller 341 is coupled, third section 360 (e.g., wound material), and fourth section 365 (e.g., tubular material) to which distal impeller 342 is coupled. The drive cable sections all have the same inner diameter, so that lumen has a constant inner diameter. The drive cable sections can be secured to each other using known attachment techniques. A distal end of fourth section 365 extends to a distal region of the working portion, allowing the working portion to be, for example, advanced over a guidewire for positioning the working portion. In this embodiment the second and fourth sections can be stiffer than first and third sections. For example, second and fourth can be tubular and first and third sections can be wound material to impart less stiffness.

Pump portion 340 includes proximal expandable scaffold 343 and distal expandable scaffold 344, each of which extends radially outside of one of the impellers. The expandable scaffolds have distal and proximal ends that also extend axially beyond distal and proximal ends of the impellers, which can be seen in FIGS. 3B-3D. Coupled to the two expandable scaffolds is blood conduit 356, which has a proximal end 353 and a distal end 352. The two expandable scaffolds each include a plurality of proximal struts and a plurality of distal struts. The proximal struts in proximal expandable scaffold 343 extend to and are secured to shaft section 345, which is coupled to bearing 361, through which the drive cable extends and is configured and sized to rotate. The distal struts of proximal expandable scaffold 343 extend to and are secured to a proximal region (to a proximal end in this case) of central tubular member 346, which is disposed axially in between the expandable members. The proximal end of central tubular member 346 is coupled to bearing 349, as shown in FIG. 3C, through which the drive cable extends and rotates. The proximal struts extend axially from distal expandable scaffold 344 to and are secured to a distal region (to a distal end in this case) of central tubular member 346. Bearing 350 is also coupled to the distal region of central tubular member 346, as is shown in FIG. 3D. The drive cable extends through and rotates relative to bearing 350. Distal struts extend from the distal expandable scaffold extend to and are secured to shaft section 347 (see FIG. 3A), which can be considered part of the distal tip. Shaft section 347 is coupled to bearing 351 (see FIG. 3D), through which the drive cable extends and rotates relative to. The distal tip also includes bearing 366 (see FIG. 3D), which can be a thrust bearing. Working portion 340 can be similar to or the same, in some aspects, to working portion 1104, even if not explicitly included in the description. In this embodiment, conduit 356 extends at least as far as ends of the impeller, unlike in working portion 1104. Either embodiment can be modified so that the conduit extends to a position as set forth in the other embodiment. In some embodiments, section 360 can be a tubular section instead of wound.

In alternative embodiments, at least a portion of any of the impellers herein may extend outside of the fluid lumen. For example, only a portion of an impeller may extend beyond an end of the fluid lumen in either the proximal or distal direction. In some embodiments, a portion of an impeller that extends outside of the fluid lumen is a proximal portion of the impeller, and includes a proximal end (e.g., see the proximal impeller in FIG. 2). In some embodiments, the portion of the impeller that extends outside of the fluid lumen is a distal portion of the impeller, and includes a distal end (e.g., see the distal impeller in FIG. 2). When the disclosure herein refers to impellers that extend outside of the fluid lumen (or beyond an end), it is meant to refer to relative axial positions of the components, which can be most easily seen in side views or top views, such as in FIG. 2.

A second impeller at another end of the fluid lumen may not, however, extend beyond the fluid lumen. For example, an illustrative alternative design can include a proximal impeller that extends proximally beyond a proximal end of the fluid lumen (like the proximal impeller in FIG. 2), and the fluid lumen does not extend distally beyond a distal end of a distal impeller (like in FIG. 3B). Alternatively, a distal end of a distal impeller can extend distally beyond a distal end of the fluid lumen, but a proximal end of a proximal impeller does not extend proximally beyond a proximal end of the fluid lumen. In any of the pump portions herein, none of the impellers may extend beyond ends of the fluid lumen.

While specific exemplary locations may be shown herein, the fluid pumps may be able to be used in a variety of locations within a body. Some exemplary locations for placement include placement in the vicinity of an aortic valve or pulmonary valve, such as spanning the valve and positioned on one or both sides of the valve, and in the case of an aortic valve, optionally including a portion positioned in the ascending aorta. In some other embodiments, for example, the pumps may be, in use, positioned further downstream, such as being disposed in a descending aorta.

FIG. 4 illustrates an exemplary placement of pump portion 1104 from catheter blood pump 1000 from FIG. 2. Once difference shown in FIG. 4 is that the conduit extends at least as far as the ends of the impellers, like in FIGS. 3A-3D. FIG. 4 shows pump portion 1104 in a deployed configuration, positioned in place across an aortic valve. Pump portion 1104 can be delivered as shown via, for example without limitation, femoral artery access (a known access procedure). While not shown for clarity, system 1000 can also include an outer sheath or shaft in which working portion 1104 is disposed during delivery to a location near an aortic valve. The sheath or shaft can be moved proximally (towards the ascending aorta “AA” and away from left ventricle “LV”) to allow for deployment and expansion of working portion 1104. For example, the sheath can be withdrawn to allow for expansion of second expandable scaffold 1110, with continued proximal movement allowing first expandable scaffold 1108 to expand.

In this embodiment, second expandable scaffold 1110 has been expanded and positioned in a deployed configuration such that distal end 1125 is in the left ventricle “LV,” and distal to aortic valve leaflets “VL,” as well as distal to the annulus. Proximal end 1124 has also been positioned distal to leaflets VL, but in some methods proximal end 1124 may extend slightly axially within the leaflets VL. This embodiment is an example of a method in which at least half of the second expandable member 1110 is within the left ventricle, as measured along its length (measured along the longitudinal axis). And as shown, this is also an example of a method in which the entire second expandable member 1110 is within the left ventricle. This is also an example of a method in which at least half of second impeller 1118 is positioned within the left ventricle, and also an embodiment in which the entire second impeller 1118 is positioned within the left ventricle.

Continued retraction of an outer shaft or sheath (and/or distal movement of working end 1104 relative to an outer sheath or shaft) continues to release conduit 1112, until central region 1113 is released and deployed. The expansion of expandable scaffolds 1108 and 1110 causes blood conduit 1112 to assume a more open configuration, as shown in FIG. 4. Thus, while in this embodiment conduit 1112 does not have the same self-expanding properties as the expandable scaffolds, the conduit will assume a deployed, more open configuration when the working end is deployed. At least a portion of central region 1113 of conduit 1112 is positioned at an aortic valve coaptation region and engages leaflets. In FIGS. 3, there is a short length of central region 1113 that extends distally beyond the leaflets VL, but at least some portion of central region 1113 is axially within the leaflets.

Continued retraction of an outer shaft or sheath (and/or distal movement of working end 1104 relative to an outer sheath or shaft) deploys first expandable member 1108. In this embodiment, first expandable scaffold 1108 has been expanded and positioned (as shown) in a deployed configuration such that proximal end 1121 is in the ascending aorta AA, and proximal to leaflets “VL.” Distal end 1122 has also been positioned proximal to leaflets VL, but in some methods distal end 1122 may extend slightly axially within the leaflets VL. This embodiment is an example of a method in which at least half of first expandable member 1110 is within the ascending aorta, as measured along its length (measured along the longitudinal axis). And as shown, this is also an example of a method in which the entire first expandable member 1110 is within the AA. This is also an example of a method in which at least half of first impeller 1116 is positioned within the AA, and also an embodiment in which the entire first impeller 1116 is positioned within the AA.

At any time during or after deployment of pump portion 1104, the position of the pump portion can be assessed in any way, such as under fluoroscopy. The position of the pump portion can be adjusted at any time during or after deployment. For example, after second expandable scaffold 1110 is released but before first expandable member 1108 is released, pump portion 1104 can be moved axially (distally or proximally) to reposition the pump portion. Additionally, for example, the pump portion can be repositioned after the entire working portion has been released from a sheath to a desired final position.

It is understood that the positions of the components (relative to the anatomy) shown in FIG. 4 are considered exemplary final positions for the different components of working portion 1104, even if there was repositioning that occurred after initial deployment.

The one or more expandable members herein can be configured to be, and can be expanded in a variety of ways, such as via self-expansion, mechanical actuation (e.g., one or more axially directed forces on the expandable member, expanded with a separate balloon positioned radially within the expandable member and inflated to push radially outward on the expandable member), or a combination thereof.

Expansion as used herein refers generally to reconfiguration to a larger profile with a larger radially outermost dimension (relative to the longitudinal axis), regardless of the specific manner in which the one or more components are expanded. For example, a stent that self-expands and/or is subject to a radially outward force can “expand” as that term is used herein. A device that unfurls or unrolls can also assume a larger profile, and can be considered to expand as that term is used herein.

The impellers can similarly be adapted and configured to be, and can be expanded in a variety of ways depending on their construction. For examples, one or more impellers can, upon release from a sheath, automatically revert to or towards a different larger profile configuration due to the material(s) and/or construction of the impeller design (see, for example, U.S. Pat. No. 6,533,716, or U.S. Pat. No. 7,393,181, both of which are incorporated by reference herein for all purposes). Retraction of an outer restraint can thus, in some embodiments, allow both the expandable member and the impeller to revert naturally to a larger profile, deployed configuration without any further actuation.

As shown in the example in FIG. 4, the working portion includes first and second impellers that are spaced on either side of an aortic valve, each disposed within a separate expandable member. This is in contrast to some designs in which a working portion includes a single elongate expandable member. Rather than a single generally tubular expandable member extending all the way across the valve, working end 1104 includes a conduit 1112 extending between expandable members 1108 and 1110. The conduit is more flexible and deformable than the expandable baskets, which can allow for more deformation of the working portion at the location of the leaflets than would occur if an expandable member spanned the aortic valve leaflets. This can cause less damage to the leaflets after the working portion has been deployed in the subject. It should also be understood that, while the embodiment of FIG. 4 shows two impellers, in other embodiments this blood pump can include only a single impeller, such as only a proximal impeller or only a distal impeller.

Additionally, forces on a central region of a single expandable member from the leaflets might translate axially to other regions of the expandable member, perhaps causing undesired deformation of the expandable member at the locations of the one or more impellers. This may cause the outer expandable member to contact the impeller, undesirably interfering with the rotation of the impeller. Designs that include separate expandable members around each impeller, particularly where each expandable member and each impeller are supported at both ends (i.e., distal and proximal), result in a high level of precision in locating the impeller relative to the expandable member. Two separate expandable members may be able to more reliably retain their deployed configurations compared with a single expandable member.

As described herein above, it may be desirable to be able to reconfigure the working portion so that it can be delivered within a 9 F sheath and still obtain high enough flow rates when in use, which is not possible with some products currently in development and/or testing. For example, some products are too large to be able to be reconfigured to a small enough delivery profile, while some smaller designs may not be able to achieve the desired high flow rates. An exemplary advantage of the examples in FIGS. 1, 2, 3A-3D and 4 is that, for example, the first and second impellers can work together to achieve the desired flow rates, and by having two axially spaced impellers, the overall working portion can be reconfigured to a smaller delivery profile than designs in which a single impeller is used to achieve the desired flow rates. These embodiments thus use a plurality of smaller, reconfigurable impellers that are axially spaced to achieve both the desired smaller delivery profile as well as to achieve the desired high flow rates.

The embodiment herein can thus achieve a smaller delivery profile while maintaining sufficiently high flow rates, while creating a more deformable and flexible central region of the working portion, the exemplary benefits of which are described above (e.g., interfacing with delicate valve leaflets).

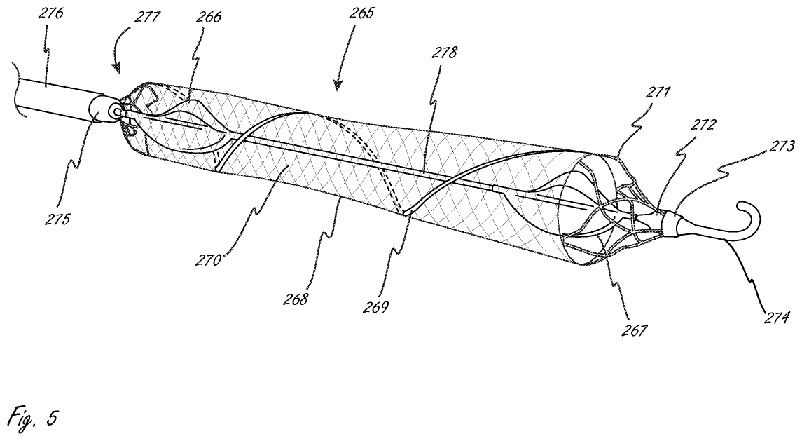

FIG. 5 illustrates a working portion that is similar to the working portion shown in FIG. 1. Working portion 265 includes proximal impeller 266, distal impeller 267, both of which are coupled to drive shaft 278, which extends into distal bearing housing 272. There is a similar proximal bearing housing at the proximal end of the working portion. Working portion also includes expandable scaffold or member, referred to 270 generally, and blood conduit 268 that is secured to the expandable member and extends almost the entire length of expandable member. Expandable member 270 includes distal struts 271 that extend to and are secured to strut support 273, which is secured to distal tip 273. Expandable member 270 also includes proximal struts there are secured to a proximal strut support. It should also be understood that, while the embodiment of FIG. 5 shows two impellers, in other embodiments this blood pump can include only a single impeller, such as only a proximal impeller or only a distal impeller.

All features similar to that shown in FIG. 1 are incorporated by reference for all purposes into this embodiment even if not explicitly stated. Expandable member 265 also includes helical tension member 269 that is disposed along the periphery of the expandable member, and has a helical configuration when the expandable member is in the expanded configuration as shown. The helical tension member 269 is disposed and adapted to induce rotation wrap upon collapse. Working portion 265 can be collapsed from the shown expanded configuration while simultaneously rotating one or both impellers at a relatively slow speed to facilitate curled collapse of the impellers due to interaction with the expandable member. Helical tension member 269 (or a helical arrangement of expandable member cells) will act as a collective tension member and is configured so that when the expandable basket is pulled in tension along its length to collapse (such as by stretching to a much greater length, such as approximately doubling in length) tension member 269 is pulled into a straighter alignment, which causes rotation/twisting of the desired segment(s) of the expandable member during collapse, which causes the impeller blades to wrap radially inward as the expandable member and blades collapse. An exemplary configuration of such a tension member would have a curvilinear configuration when in helical form that is approximately equal to the maximum length of the expandable member when collapsed. In alternative embodiments, only the portion(s) of the expandable member that encloses a collapsible impeller is caused to rotate upon collapse.

There are alternative ways to construct the working portion to cause rotation of the expandable member upon collapse by elongation (and thus cause wrapping and collapse of the impeller blades). Any expandable member can be constructed with this feature, even in dual-impeller designs. For example, with an expandable member that includes a plurality of “cells,” as that term is commonly known (e.g., a laser cut elongate member), the expandable member may have a plurality of particular cells that together define a particular configuration such as a helical configuration, wherein the cells that define the configuration have different physical characteristics than other cells in the expandable member. In some embodiments the expandable member can have a braided construction, and the twist region may constitute the entire group of wires, or a significant portion (e.g., more than half), of the braided wires. Such a twisted braid construction may be accomplished, for example, during the braiding process, such as by twisting the mandrel that the wires are braided onto as the mandrel is pulled along, especially along the length of the largest-diameter portion of the braided structure. The construction could also be accomplished during a second operation of the construction process, such as mechanically twisting a braided structure prior to heat-setting the wound profile over a shaped mandrel.

Any of the blood conduits herein act to, are configured to, and are made of material(s) that create a fluid lumen therein between a first end (e.g., distal end) and a second end (e.g., proximal end). Fluid flows into the inflow region, through the fluid lumen, and then out of an outflow region. Flow into the inflow region may be labeled herein as “I,” and flow out at the outflow region may be labeled “O.” Any of the conduits herein can be impermeable. Any of the conduits herein can alternatively be semipermeable. Any of the conduits herein may also be porous, but will still define a fluid lumen therethrough. In some embodiments the conduit is a membrane, or other relatively thin layered member. Any of the conduits herein, unless indicated to the contrary, can be secured to an expandable member such that the conduit, where is it secured, can be radially inside and/or outside of the expandable member. For example, a conduit may extend radially within the expandable member so that inner surface of the conduit is radially within the expandable member where it is secured to the expandable member.

Any of the expandable scaffolds or member(s) herein may be constructed of a variety of materials and in a variety of ways. For example, the expandable member may have a braided construction, or it can be formed by laser machining. The material can be deformable, such as nitinol. The expandable member can be self-expanding or can be adapted to be at least partially actively expanded.

In some embodiments, the expandable scaffold or member is adapted to self-expand when released from within a containing tubular member such as a delivery catheter, a guide catheter or an access sheath. In some alternative embodiments, the expandable member is adapted to expand by active expansion, such as action of a pull-rod that moves at least one of the distal end and the proximal end of the expandable member toward each other. In alternative embodiments, the deployed configuration can be influenced by the configuration of one or more expandable structures. In some embodiments, the one or more expandable members can be deployed, at least in part, through the influence of blood flowing through the conduit. Any combination of the above mechanisms of expansion may be used.

The blood pumps and fluid movement devices, system and methods herein can be used and positioned in a variety of locations within a body. While specific examples may be provided herein, it is understood that that the working portions can be positioned in different regions of a body than those specifically described herein.

In any of the embodiments herein in which the catheter blood pump includes a plurality of impellers, the device can be adapted such that the impellers rotate at different speeds. FIG. 6A illustrates a medical device that includes gearset 1340 coupled to both inner drive member 1338 and outer drive member 1336, which are in operable communication with distal impeller 1334 and proximal impeller 1332, respectively. The device also includes motor 1342, which drives the rotation of inner drive member 1338. Inner drive member 1338 extends through outer drive member 1336. Activation of the motor 1332 causes the two impellers to rotate at different speeds due to an underdrive or overdrive ratio. Gearset 1340 can be adapted to drive either the proximal or distal impeller faster than the other. Any of the devices herein can include any of the gearsets herein to drive the impellers at different speeds.

FIG. 6B illustrates a portion of an alternative embodiment of a dual impeller device (1350) that is also adapted such that the different impellers rotate at different speeds. Gearset 1356 is coupled to both inner drive member 1351 and outer drive member 1353, which are coupled to distal impeller 1352 and proximal impeller 1354, respectively. The device also includes a motor like in FIG. 6A. FIGS. 6A and 6B illustrate how a gearset can be adapted to drive the proximal impeller slower or faster than the distal impeller.

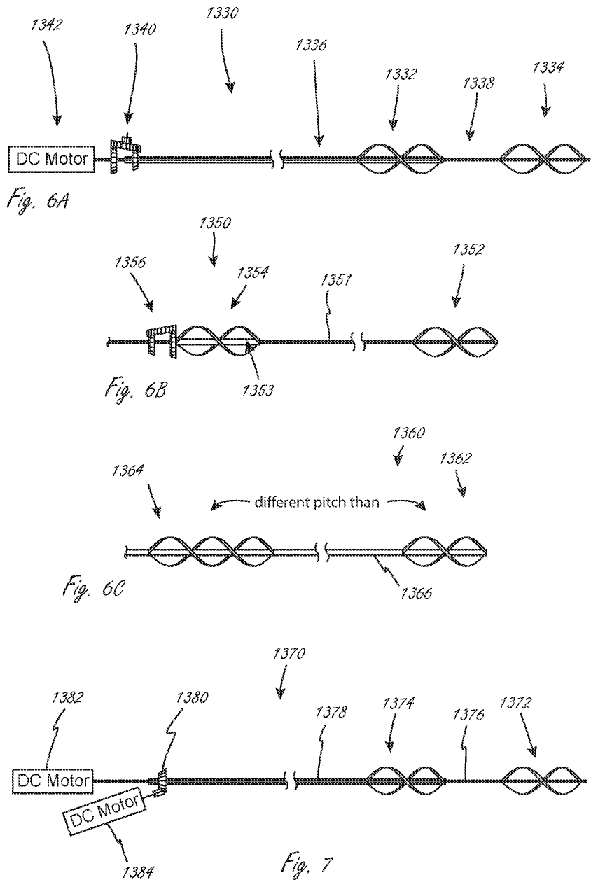

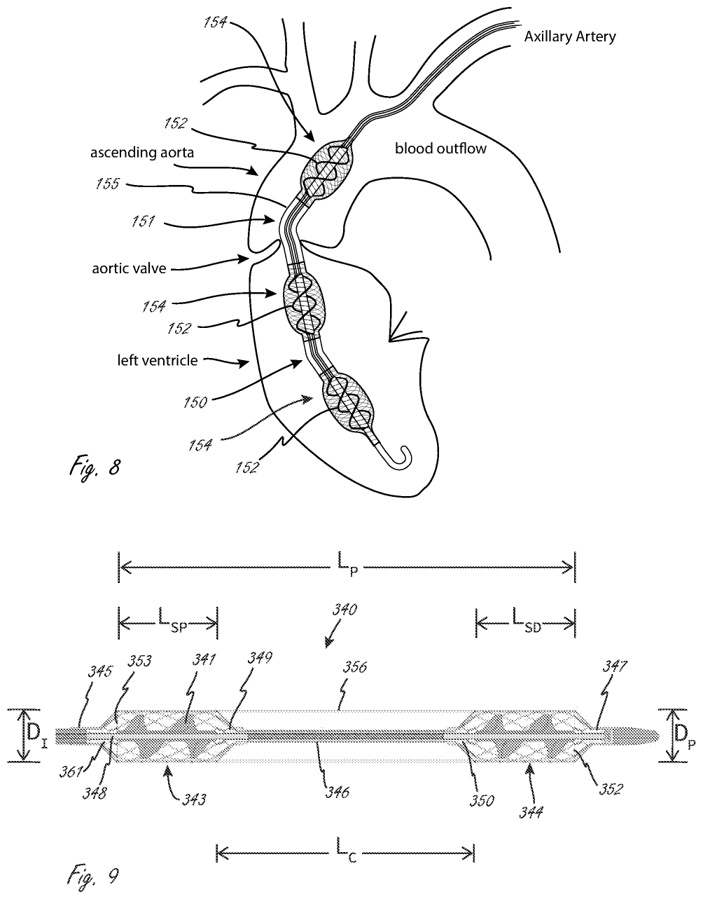

FIG. 7 illustrates an exemplary alternative embodiment of fluid pump 1370 that can rotate first and second impellers at different speeds. First motor 1382 drives cable 1376, which is coupled to distal impeller 1372, while second motor 1384 drives outer drive member 1378 (via gearset 1380), which is coupled to proximal impeller 1374. Drive cable 1376 extends through outer drive member 1378. The motors can be individually controlled and operated, and thus the speeds of the two impellers can be controlled separately. This system setup can be used with any system herein that includes a plurality of impellers.

In some embodiments, a common drive mechanism (e.g., cable and/or shaft) can drive the rotation of two (or more) impellers, but the blade pitch of the two impellers (angle of rotational curvature) can be different, with the distal or proximal impeller having a steeper or more gradual angle than the other impeller. This can produce a similar effect to having a gearset. FIG. 6C shows a portion of a medical device (1360) that includes common drive cable 1366 coupled to proximal impeller 1364 and distal impeller 1362, and to a motor not shown. The proximal impellers herein can have a greater or less pitch than the distal impellers herein. Any of the working portions (or distal portions) herein with a plurality of impellers can be modified to include first and second impellers with different pitches.

In any of the embodiments herein, the pump portion may have a compliant or semi-compliant (referred to generally together as “compliant”) exterior structure. In various embodiments, the compliant portion is pliable. In various embodiments, the compliant portion deforms only partially under pressure. For example, the central portion of the pump may be formed of a compliant exterior structure such that it deforms in response to forces of the valve. In this manner the exterior forces of the pump on the valve leaflets are reduced. This can help prevent damage to the valve at the location where it spans the valve.

FIG. 8 illustrates an exemplary embodiment of a pump portion that includes first, second and third axially spaced impellers 152, each of which is disposed within an expandable member 154. Conduit 155 can extend along the length of the pump portion, as in described in various embodiments herein, which can help create and define the fluid lumen. In alternative embodiments, however, the first, second, and third impellers may be disposed within a single expandable member, similar to that shown in FIG. 1. In FIG. 8, a fluid lumen extends from a distal end to a proximal end, features of which are described elsewhere herein. The embodiment in FIG. 8 can include any other suitable feature, including methods of use, described herein.

The embodiment in FIG. 8 is also an example of an outer housing having at least one bend formed therein between a proximal impeller distal end and a distal impeller proximal end, such that a distal region of the housing distal to the bend is not axially aligned with a proximal region of the housing proximal to the bend along an axis. In this embodiment there are two bends 150 and 151 formed in the housing, each one between two adjacent impellers.

In a method of use, a bend formed in a housing can be positioned to span a valve, such as the aortic valve shown in FIG. 8. In this method of placement, a central impeller and distal-most impeller are positioned in the left ventricle, and a proximal-most impeller is positioned in the ascending aorta. Bend 151 is positioned just downstream to the aortic valve.

A bend such as bend 150 or 151 can be incorporated into any of the embodiments or designs herein. The bend may be a preformed angle or may be adjustable in situ.

In any of the embodiments herein, unless indicated to the contrary, the outer housing can have a substantially uniform diameter along its length.

In FIG. 8, the pump is positioned via the axillary artery, which is an exemplary method of accessing the aortic valve, and which allows the patient to walk and be active with less interruption. Any of the devices herein can be positioned via the axillary artery. It will be appreciated from the description herein, however, that the pump may be introduced and tracked into position in various manners including a femoral approach over the aortic arch.

One aspect of the disclosure is a catheter blood pump that includes a distal impeller axially spaced from a proximal impeller. Distal and proximal impellers may be axially spaced from each other. For example, the distal and proximal impellers may be connected solely by their individual attachment to a common drive mechanism. This is different from a single impeller having multiple blade rows or sections. A distal impeller as that phrase is used herein does not necessarily mean a distal-most impeller of the pump, but can refer generally to an impeller that is positioned further distally than a proximal impeller, even if there is an additional impeller than is disposed further distally than the distal impeller. Similarly, a proximal impeller as that phrase is used herein does not necessarily mean a proximal-most impeller of the pump, but can refer generally to an impeller that is positioned further proximally than a proximal impeller, even if there is an additional impeller than is disposed further proximally than the proximal impeller. Axial spacing (or some derivative thereof) refers to spacing along the length of a pump portion, such as along a longitudinal axis of the pump portion, even if there is a bend in the pump portion. In various embodiments, each of the proximal and distal impellers are positioned within respective housings and configured to maintain a precise, consistent tip gap, and the span between the impellers has a relatively more flexible (or completely flexible) fluid lumen. For example, each of the impellers may be positioned within a respective housing having relatively rigid outer wall to resist radial collapse. The sections between the impellers may be relatively rigid, in some embodiments the section is held open primarily by the fluid pressure within.

Although not required for the embodiments therein, there may be advantages to having a minimum axial spacing between a proximal impeller and a distal impeller. For example, a pump portion may be delivered to a target location through parts of the anatomy that have relatively tight bends, such as, for example, an aorta, and down into the aortic valve. For example, a pump portion may be delivered through a femoral artery access and to an aortic valve. It can be advantageous to have a system that is easier to bend so that it is easier to deliver the system through the bend(s) in the anatomy. Some designs where multiple impellers are quite close to each other may make the system, along the length that spans the multiple impellers, relatively stiff along that entire length that spans the multiple impellers. Spacing the impellers apart axially, and optionally providing a relatively flexible region in between the impellers, can create a part of the system that is more flexible, is easier to bend, and can be advanced through the bends more easily and more safely. An additional exemplary advantage is that the axial spacing can allow for a relatively more compliant region between the impellers, which can be positioned at, for example, the location of a valve (e.g., an aortic valve). Furthermore, there are other potential advantages and functional differences between the various embodiments herein and typical multistage pumps. A typical multistage pump includes rows of blades (sometimes referred to as impellers) in close functional spacing such that the rows of blades act together as a synchronized stage. One will appreciate that the flow may separate as it passes through the distal impeller. In various embodiments as described herein, distal and proximal impellers can be spaced sufficiently apart such that the flow separation from the distal impeller is substantially reduced (i.e., increased flow reattachment) and the localized turbulent flow is dissipated before the flow enters the proximal impeller.

In any of the embodiments or in any part of the description herein that include a distal impeller and a proximal impeller, the axial spacing between a distal end of the proximal impeller and a proximal end of the distal impeller can be from 1.5 cm to 25 cm (inclusive) along a longitudinal axis of the pump portion, or along a longitudinal axis of a housing portion that includes a fluid lumen. The distance may be measured when the pump portion, including any impellers, is in an expanded configuration. This exemplary range can provide the exemplary flexibility benefits described herein as the pump portion is delivered through curved portions of the anatomy, such as, for example, an aortic valve via an aorta. FIG. 9 (shown outside a patient in an expanded configuration) illustrates length Lc, which illustrates an axial spacing between impellers, and in some embodiments may be from 1.5 cm to 25 cm as set forth herein. In embodiments in which there may be more than two impellers, any two adjacent impellers (i.e., impellers that do not have any other rotating impeller in between them) may be spaced axially by any of the axial spacing distances described herein.

While some embodiments include a proximal impeller distal end that is axially spaced 1.5 cm to 25 cm from a distal impeller proximal end along an axis, the disclosure herein also includes any axial spacings that are subranges within that general range of 1.5 cm to 25 cm. That is, the disclosure includes all ranges that have any lower limit from 1.5 and above in that range, and all subranges that have any upper limit from 25 cm and below. The examples below provide exemplary subranges. In some embodiments, a proximal impeller distal end is axially spaced 1.5 cm to 20 cm from a distal impeller proximal end along an axis, 1.5 cm to 15 cm, 1.5 cm to 10 cm, 1.5 cm to 7.5 cm, 1.5 cm to 6 cm, 1.5 cm to 4.5 cm, 1.5 cm to 3 cm. In some embodiments the axial spacing is 2 cm to 20 cm, 2 cm to 15 cm, 2 cm to 12 cm, 2 cm to 10 cm, 2 cm to 7.5 cm, 2 cm to 6 cm, 2 cm to 4.5 cm, 2 cm to 3 cm. In some embodiments the axial spacing is 2.5 cm to 15 cm, 2.5 cm to 12.5 cm, 2.5 cm to 10 cm, 2.5 cm to 7.5 cm, or 2.5 cm to 5 cm (e.g., 3 cm). In some embodiments the axial spacing is 3 cm to 20 cm, 3 cm to 15 cm, 3 cm to 10 cm, 3 cm to 7.5 cm, 3 cm to 6 cm, or 3 cm to 4.5 cm. In some embodiments the axial spacing is 4 cm to 20 cm, 4 cm to 15 cm, 4 cm to 10 cm, 4 cm to 7.5 cm, 4 cm to 6 cm, or 4 cm to 4.5 cm. In some embodiments the axial spacing is 5 cm to 20 cm, 5 cm to 15 cm, 5 cm to 10 cm, 5 cm to 7.5 cm, or 5 cm to 6 cm. In some embodiments the axial spacing is 6 cm to 20 cm, 6 cm to 15 cm, 6 cm to 10 cm, or 6 cm to 7.5 cm. In some embodiments the axial spacing is 7 cm to 20 cm, 7 cm to 15 cm, or 7 cm to 10 cm. In some embodiments the axial spacing is 8 cm to 20 cm, 8 cm to 15 cm, or 8 cm to 10 cm. In some embodiments the axial spacing is 9 cm to 20 cm, 9 cm to 15 cm, or 9 cm to 10 cm. In various embodiments, the fluid lumen between the impellers is relatively unsupported.

In any of the embodiments herein the one or more impellers may have a length, as measured axially between an impeller distal end and an impeller proximal end (shown as “L_{SD}” and “L_{SP}”, respectively, in FIG. 9), from 0.5 cm to 10 cm, or any subrange thereof. The examples below provide exemplary subranges. In some embodiments the impeller axial length is from 0.5 cm to 7.5 cm, from 0.5 cm to 5 cm, from 0.5 cm to 4 cm, from 0.5 cm to 3 cm, from 0.5 cm to 2, or from 0.5 cm to 1.5 cm. In some embodiments the impeller axial length is from 0.8 cm to 7.5 cm, from 0.8 cm to 5 cm, from 0.8 cm to 4 cm, from 0.8 cm to 3 cm, from 0.8 cm to 2 cm, or from 0.8 cm to 1.5 cm. In some embodiments the impeller axial length is from 1 cm to 7.5 cm, from 1 cm to 5 cm, from 1 cm to 4 cm, from 1 cm to 3 cm, from 1 cm to 2 cm, or from 1 cm to 1.5 cm. In some embodiments the impeller axial length is from 1.2 cm to 7.5 cm, from 1.2 cm to 5 cm, from 1.2 cm to 4 cm, from 1.2 cm to 3 cm, from 1.2 to 2 cm, or from 1.2 cm to 1.5 cm. In some embodiments the impeller axial length is from 1.5 cm to 7.5 cm, from 1.5 cm to 5 cm, from 1.5 cm to 4 cm, from 1.5 cm to 3 cm, or from 1.5 cm to 2 cm. In some embodiments the impeller axial length is from 2 cm to 7.5 cm, from 2 cm to 5 cm, from 2 cm to 4 cm, or from 2 cm to 3 cm. In some embodiments the impeller axial length is from 3 cm to 7.5 cm, from 3 cm to 5 cm, or from 3 cm to 4 cm. In some embodiments the impeller axial length is from 4 cm to 7.5 cm, or from 4 cm to 5 cm.

In any of the embodiments herein the fluid lumen can have a length from a distal end to a proximal end, shown as length Lp in FIG. 9. In some embodiments the fluid lumen length Lp is from 4 cm to 40 cm, or any subrange therein. For example, in some embodiments the length Lp can be from 4 cm to 30 cm, from 4 cm to 20 cm, from 4 cm to 18 cm, from 4 cm to 16 cm, from 4 cm to 14 cm, from 4 cm to 12 cm, from 4 cm to 10 cm, from 4 cm to 8 cm, from 4 cm to 6 cm.

In any of the embodiments herein the housing can have a deployed diameter, at least the location of an impeller (and optionally at a location between impellers), shown as dimension Dp in FIG. 9. In some embodiments Dp can be from 0.3 cm to 1.5 cm, or any subrange therein. For example, Dp may be from 0.4 cm to 1.4 cm, from 0.4 cm to 1.2 cm, from 0.4 cm to 1.0 cm, from 0.4 cm to 0.8 cm, or from 0.4 cm to 0.6 cm. In some embodiments, Dp may be from 0.5 cm to 1.4 cm, from 0.5 cm to 1.2 cm, from 0.5 cm to 1.0 cm, from 0.5 cm to 0.8 cm, or from 0.5 cm to 0.6 cm. In some embodiments Dp may be from 0.6 cm to 1.4 cm, from 0.6 cm to 1.2 cm, from 0.6 cm to 1.0 cm, or from 0.6 cm to 0.8 cm. In some embodiments Dp may be from 0.7 cm to 1.4 cm, from 0.7 cm to 1.2 cm, from 0.7 cm to 1.0 cm, or from 0.7 cm to 0.8 cm.

In any of the embodiments herein an impeller can have a deployed diameter, shown as dimension Di in FIG. 9. In some embodiments Di can be from 1 mm-30 mm, or any subrange therein. For example, in some embodiments Di may be from 1 mm-15 mm, from 2 mm-12 mm, from 2.5 mm-10 mm, or 3 mm-8 mm.

In any of the embodiments herein, a tip gap exists between an impeller outer diameter and a fluid lumen inner diameter. In some embodiments the tip gap can be from 0.01 mm-1 mm, such as 0.05 mm to 0.8 mm, or such as 0.1 mm-0.5 mm.

In any of the embodiments herein that includes multiple impellers, the axial spacing between impellers (along the length of the pump portion, even if there is a bend in the pump portion) can be from 2 mm to 100 mm, or any combination of upper and lower limits inclusive of 5 and 100 mm (e.g., from 10 mm-80 mm, from 15 mm-70 mm, from 20 mm-50 mm, 2 mm-45 mm, etc.).

Any of the pump portions herein that include a plurality of impellers may also include more than two impellers, such as three, four, or five impellers (for example).

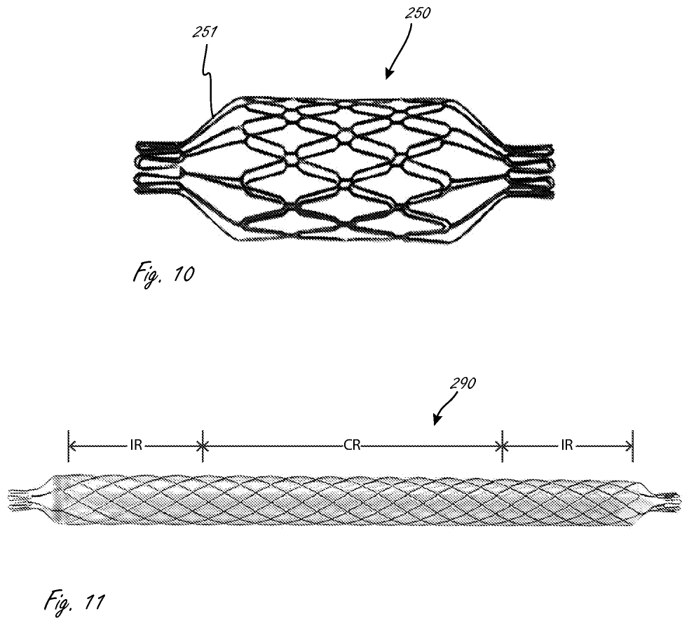

FIG. 10 illustrates an expandable scaffold 250 that may be one of at least two expandable scaffolds of a pump portion, such as the expandable scaffolds in FIGS. 3A-3D, wherein each expandable scaffold at least partially surrounds an impeller. The scaffold design in FIG. 10 has proximal struts 251 (only one labeled) extending axially therefrom. Having a separate expandable scaffold 250 for each impeller provides for the ability to have different geometries for any of the individual impellers. Additionally, this design reduces the amount of scaffold material (e.g., Nitinol) over the length of the expandable blood conduit, which may offer increased tracking when sheathed. A potential challenge with these designs may include creating a continuous membrane between the expandable scaffolds in the absence of an axially extending scaffolding material (see FIG. 3A). Any other aspect of the expandable scaffolds or members herein, such as those described in FIGS. 3A-3D, may be incorporated by reference into this exemplary design. Struts 251 may be disposed at a pump inflow or outflow. Struts 251 may be proximal struts or they may be distal struts.

FIG. 11 show an exemplary scaffold along a length of the blood conduit. Central region “CR” may be axially between proximal and distal impellers. Central region “CR” flexibility is increased relative to scaffold impeller regions “IR” due to breaks or discontinuities in the scaffold pattern in the central region. The scaffold has relatively more rigid impeller sections “IR” adjacent the central region where impellers may be disposed (not shown). The relatively increased rigidity in the impeller regions IR may help maintain tip gap and impeller concentricity. This pump scaffold pattern provides for a flexibility distribution, along its length, of a proximal section of relatively less flexibility (“IR”), a central region “CR” of relatively higher flexibility, and a distal section “IR” of relatively less flexibility than the central region. The relatively less flexible sections (i.e., the two IR regions) are where proximal and distal impellers may be disposed (not shown but other embodiments are fully incorporated herein in this regard), with a relatively more flexible region in between. Exemplary benefits of the relative flexibility in these respective sections are described elsewhere herein. FIG. 11 is an example of a scaffold that is continuous from a first end region to a second end region, even though there are breaks or discontinuities in some locations of the scaffold. There is at least one line that can be traced along a continuous structural path from a first end region to a second end region.

The following disclosure provides exemplary method steps that may be performed when using any of the blood pumps, or portions thereof, described herein. It is understood that not all of the steps need to be performed, but rather the steps are intended to be an illustrative procedure. It is also intended that, if suitable, in some instances the order of one or more steps may be different. Before use, the blood pump can be prepared for use by priming the lumens (including any annular spaces) and pump assembly with sterile solution (e.g., heparinized saline) to remove any air bubbles from any fluid lines. The catheter, including any number of purge lines, may then be connected to a console. Alternatively, the catheter may be connected to a console and/or a separate pump that are used to prime the catheter to remove air bubbles.

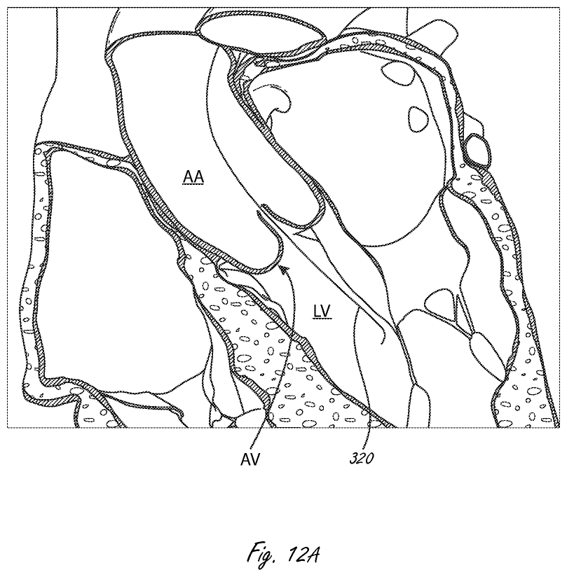

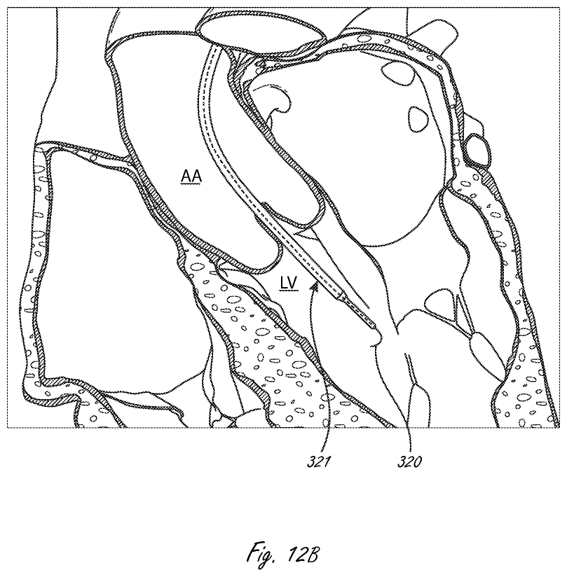

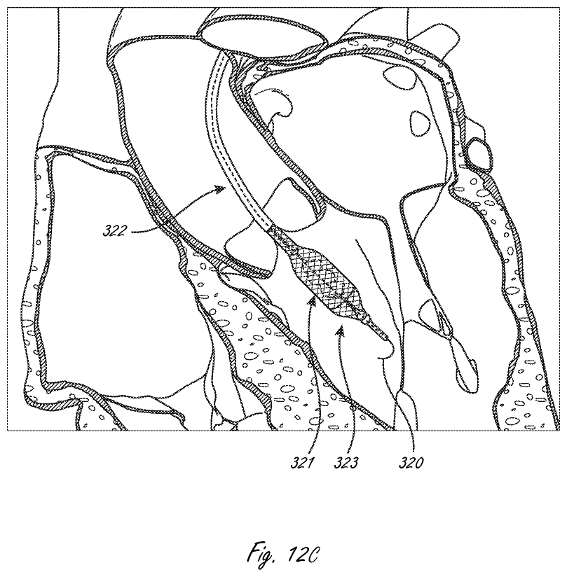

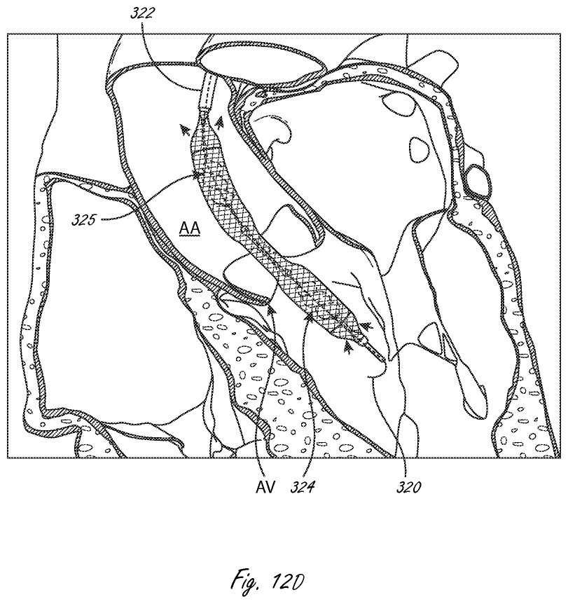

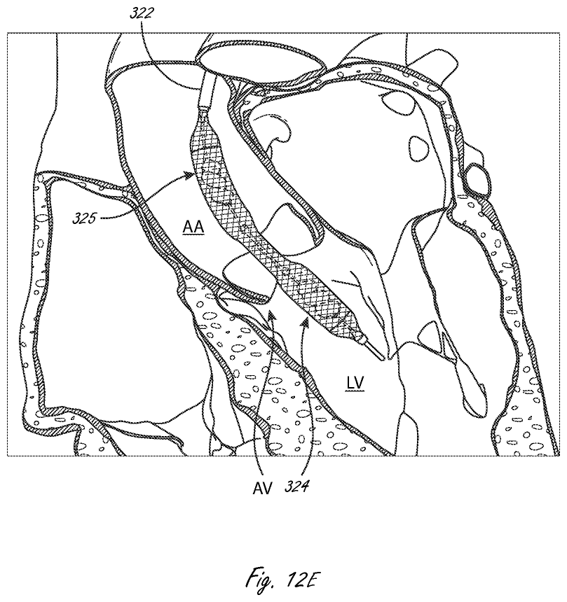

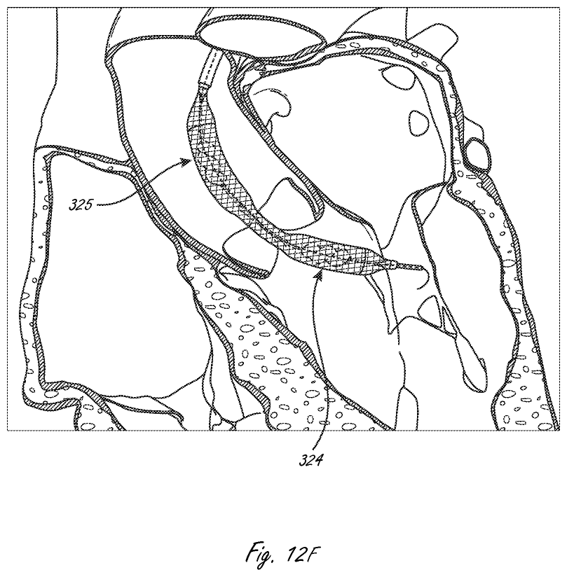

After priming the catheter, access to the patient's vasculature can be obtained (e.g., without limitation, via femoral access) using an appropriately sized introducer sheath. Using standard valve crossing techniques, a diagnostic pigtail catheter may then be advanced over a, for example, 0.035″ guide wire until the pigtail catheter is positioned securely in the target location (e.g., left ventricle). The guidewire can then be removed and a second wire 320 (e.g., a 0.018″ wire) can be inserted through the pigtail catheter. The pigtail catheter can then be removed (see FIG. 12A), and the blood pump 321 (including a catheter, catheter sheath, and pump portion within the sheath; see FIG. 12B) can be advanced over the second wire towards a target location, such as spanning an aortic valve “AV,” and into a target location (e.g., left ventricle “LV”), using, for example, one or more radiopaque markers to position the blood pump.

Once proper placement is confirmed, the catheter sheath 322 (see FIG. 12C) can be retracted, exposing first a distal region of the pump portion. In FIG. 12C a distal region of an expandable housing has been released from sheath 322 and is expanded, as is distal impeller 324. A proximal end of housing 323 and a proximal impeller are not yet released from sheath 322. Continued retraction of sheath 322 beyond the proximal end of housing 323 allows the housing 323 and proximal impeller 325 to expand (see FIG. 12D). The inflow region (shown with arrows even though the impellers are not yet rotating) and the distal impeller are in the left ventricle. The outflow (shown with arrows even though the impellers are not rotating yet) and proximal impeller are in the ascending aorta AA. The region of the outer housing in between the two impellers, which may be more flexible than the housing regions surrounding the impellers, as described in more detail herein, spans the aortic valve AV. In an exemplary operating position as shown, an inlet portion of the pump portion will be distal to the aortic valve, in the left ventricle, and an outlet of the pump portion will be proximal to the aortic valve, in the ascending aorta (“AA”).

The second wire (e.g., an 0.018″ guidewire) may then be moved prior to operation of the pump assembly (see FIG. 12E). If desired or needed, the pump portion can be deflected (active or passively) at one or more locations as described herein, as illustrated in FIG. 12F. For example, a region between two impellers can be deflected by tensioning a tensioning member that extends to a location between two impellers. The deflection may be desired or needed to accommodate the specific anatomy. As needed, the pump portion can be repositioned to achieve the intended placement, such as, for example, having a first impeller on one side of a heart valve and a second impeller on a second side of the heart valve. It is understood that in FIG. 12F, the pump portion is not in any way interfering or interacting with the mitral valve, even if it may appear that way from the figure.

As set forth above, this disclosure includes catheter blood pumps that include an expandable pump portion extending distally relative to a catheter. The pump portions include an impeller housing that includes an expandable blood conduit that defines a blood lumen. The blood conduit may include one or more scaffold sections that together may also be referred to herein as a single scaffold. In some exemplary embodiments the expandable blood conduit may include one or more of a proximal impeller scaffold, a distal impeller scaffold, and a central scaffold disposed between the proximal impeller scaffold and the distal impeller scaffold, where any combination thereof may also be referred to herein as a scaffold. Any individual proximal impeller scaffold or distal impeller scaffold may also be referred to herein as an expandable member, such as is shown in FIGS. 3A-3D. In some embodiments the expandable blood conduit may include a proximal impeller scaffold and additional scaffold extending distally therefrom, such as if the pump portion includes a proximal impeller but does not include a distal impeller. In any of the embodiments herein, a reference to a distal impeller is only by way of example, and pump portions herein need not include a distal impeller. Central scaffolds herein are generally less stiff in response to a radially inward force than a proximal scaffold, and optionally also less stiff than a distal scaffold, such as a distal impeller scaffold. Exemplary advantages of central scaffold sections that are less stiffness are set forth elsewhere herein. The blood conduit may also include a membrane coupled to the one or more scaffolds, the membrane at least partially defining the blood lumen. Membranes in this context may incorporate by reference herein the disclosure of conduits, including any feature or method of manufacturing described above. The catheter blood pumps may include an impeller disposed in a proximal region of the impeller housing, which may be a proximal impeller. The catheter blood pumps may also include a distal impeller in a distal region of the impeller housing. Exemplary impellers, including exemplary proximal and distal impellers, are set forth herein by way of example. An impeller that is at least partially within a portion of a scaffold may be described with respect to the relative position of the scaffold, such as a proximal impeller within at least a portion of a proximal scaffold, or a distal impeller within at least a portion of a distal scaffold.

When a proximal impeller is described as being within a proximal scaffold, it is understood that the proximal scaffold need not axially extend over an entire length of the impeller, as long as there is some amount of axial overlap. For example, some proximal impellers herein extend proximally from a blood conduit, and a proximal region of the proximal impeller is not surrounded by a blood conduit scaffold, while a distal region of the impeller is surrounded by scaffold. Similarly, when a distal impeller herein (if the pump includes a distal impeller) is described as being within a distal scaffold, it is understood that the distal scaffold need not axially extend over an entire length of the impeller, as long as there is some degree of axial overlap therebetween.

FIGS. 13A-17 illustrate exemplary designs for expandable scaffolds herein, which may at least partially surround an impeller that is at least partially disposed within a conduit that creates a fluid lumen. The scaffold patterns in FIGS. 13A-17 may be scaffold patterns that only extend over a particular impeller (e.g., a proximal basket or distal basket), or they may be scaffold patterns that extend over an entire blood conduit scaffold.

FIGS. 13A-17 illustrate expandable support members or scaffolds that each have an expanded configuration, wherein in the expanded configuration the support member has a plurality of continuous axially extending elements (e.g., 408, 410, 420, 430, 440) that are continuous and axially extending over at least 50% of a length of the expandable support member (e.g., Lₛ), and wherein the expandable support member includes a plurality of sets of connectors (e.g., 412/414, 409, 422/424, 432/434, 442/444) each set of connectors extending between first and second circumferentially adjacent continuous axially extending elements. In some embodiment the axially extending elements are linear or substantially linear.

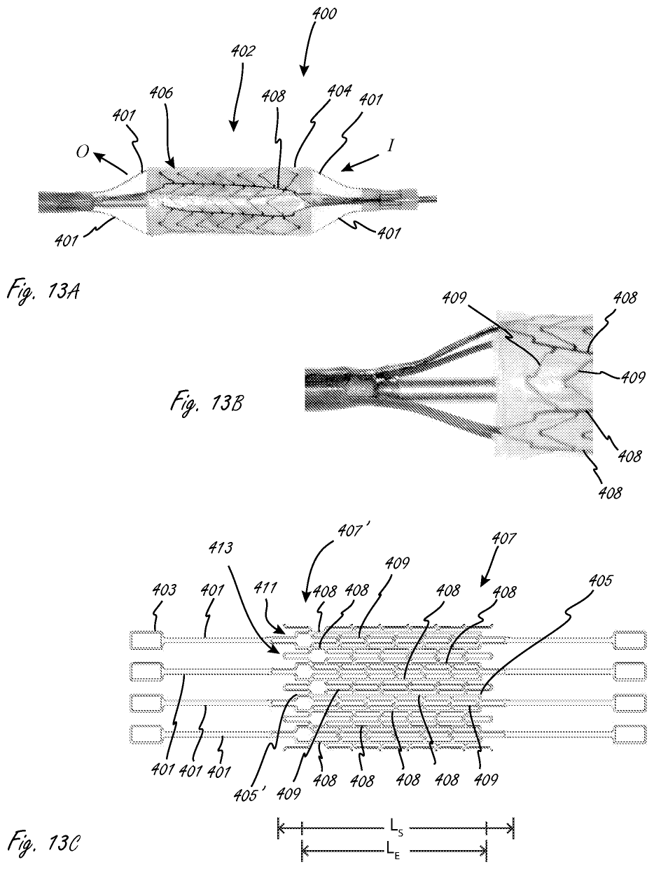

FIGS. 13A-13C illustrate an exemplary pump portion 400 or a portion thereof that comprises an expandable impeller housing 402, wherein the expandable impeller housing having a blood conduit 404, the conduit defining a blood lumen between an housing inflow “I” and a housing outflow “O”. The expandable impeller housing also includes an expandable scaffold or support member 406 at least partially surrounding an impeller (not shown in FIGS. 13A-13C) that is at least partially disposed within the conduit. FIGS. 14A-17 illustrate an expandable scaffold of the pump portion. It is understood that any expandable scaffold in any of FIGS. 13A-17 may be used in place of any expandable scaffold herein. Impeller housing 402 may illustrate the entire impeller housing, or it may only represent only a portion thereof, including only a single scaffold section, such as with any of the multi-impeller designs herein. It is thus understood that the structure shown in FIGS. 13A-C may only be a portion of the expandable housing of a pump portion. For example, a pump portion may include two of the expandable scaffold sections shown in FIGS. 13A-C, axially spaced apart, and coupled by a flexible membrane, for example.

FIGS. 13A-C illustrate an expandable impeller housing that includes a plurality of axially extending elements 408 circumferentially spaced apart around the housing 402 from adjacent axially extending elements, as shown. FIGS. 13A and 13B show an expanded configuration of the housing, while FIG. 13C illustrates a model of a flat, unexpanded configuration with unitary struts 401 extending axially therefrom, as shown. The plurality of axially extending elements may be referred to as “elements” in the context of scaffolds for simplicity, but it is understood that they are not to be considered any other type of “element” herein unless specifically indicated as such. The elements in this embodiment may be axial and linear in the housing expanded configuration. Expandable scaffold 406 also includes circumferential connectors 409 that circumferentially connect adjacent axial elements and extend from one axial element to an adjacent axial element. In this exemplary embodiment all of the connectors have the same general configuration, which includes first and second segments meeting at a rounded peak that is oriented axially (proximally or distally depending on the reference frame), otherwise stated as pointing axially. Length Ls of the scaffold and length Le of the elements is illustrated in FIG. 13C. Optional struts 401 are shown (which may be unitary with the scaffold). The axial elements 408 in this embodiment extend from a first axial element end 405 to second axial element end 405′, which extend almost the entire length of the scaffold Ls. As shown, ends 405′ of the elements (only one labeled) extend to a distal end region 407′ of the scaffold 406. End 405 extends to proximal end region 407. The pump portion also includes a transition region 411, which includes circumferential extensions of adjacent axial elements, after which they meet to form a strut 401, as shown.

FIGS. 14A (expanded) and 14B (unexpanded) illustrate an exemplary expandable scaffold 406′, which includes a plurality of axially extending elements 410. A first set of connectors 412 have “S” configurations, and a second circumferentially adjacent set of connectors 414 have inverse (reverse) “S” shapes. In the expanded configuration in FIG. 14A the axial elements 410 may be linear, or they may have a slight curvilinear configuration as shown. Scaffold 406′ includes transition region 411′, which may have similar features to the transition region 411 herein. The relevant description from any other embodiment may be incorporated with the scaffold in FIGS. 14A-B (e.g., lengths of scaffold or support member and axial elements, transition region, etc.). Some of the optional struts 413 are shown, as are ends 405/405′ of the axial elements. Scaffold 406′ may be proximal or distal scaffold, or it may extend along the length of the impeller housing.

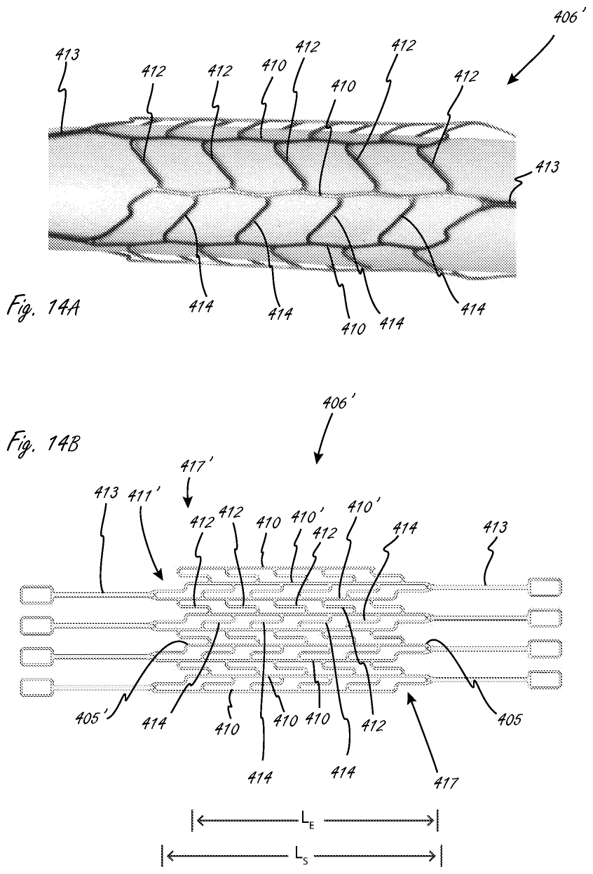

FIGS. 15A and 15B illustrate an exemplary expandable scaffold 406″ that is similar to those in FIGS. 13, 14, 16, and 17. Axially extending elements 420 are shown, adjacent ones of which are connected by circumferential connectors 422 and 424, ends of which are axially offset. A first set of connectors 422 has a general S configuration, while a second set of connectors 424 are reverse S-shaped. In this embodiment the axially extending elements 420 are curvilinear, as shown. The pattern of S and inverse-S alternates around the expandable member, as it does in the scaffolds in FIGS. 14A and 14B. Scaffold 406″ also includes a transition region 421, examples of which are described elsewhere herein. Scaffold 406″ may be proximal or distal scaffold, or it may extend along the length of the impeller housing.

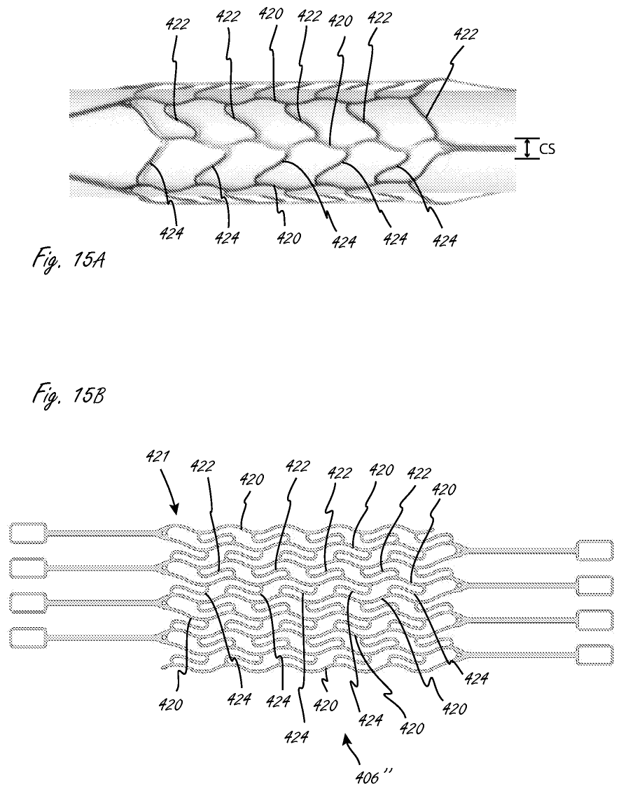

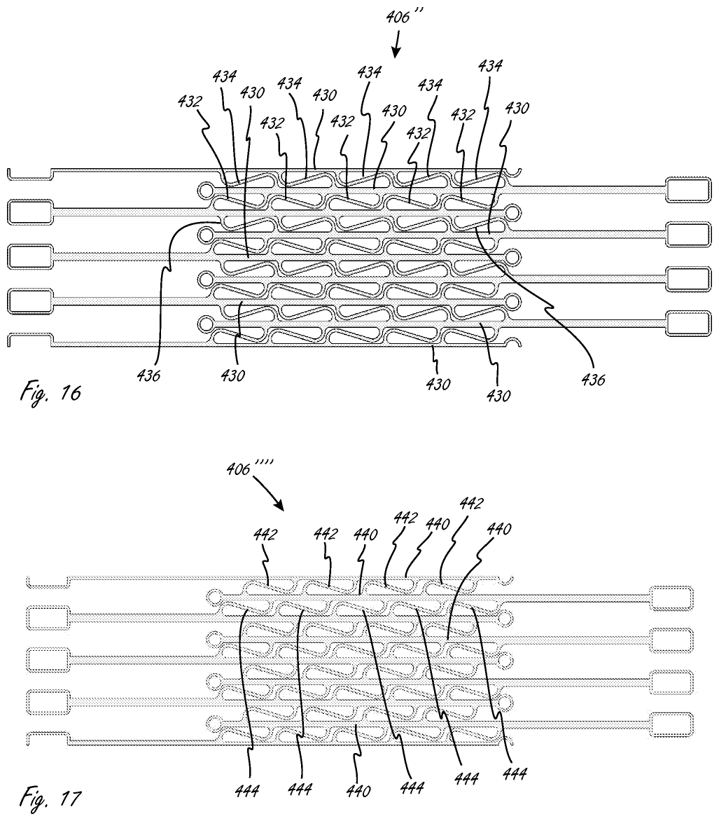

FIG. 16 illustrates a collapsed (unexpanded) configuration of an exemplary scaffold 406″′, which may have any other suitable features of any other support member or scaffold herein. Axially extending elements 430 are shown, connected by first set of S-shaped connectors 434 and a second set of inverse-S shaped connectors 432. The pattern of S and inverse-S shapes alternates circumferentially around the scaffold 406″′ as shown. Scaffold 406″′ may be proximal or distal scaffold, or it may extend along the length of the impeller housing.

FIG. 17 illustrates a collapsed (unexpanded) configuration of an exemplary scaffold 406″″, which may have any other suitable features of any other support member or scaffold herein. Axially extending elements 440 are shown, connected by inverse-S shaped connectors. All sets of the connectors in this embodiment (e.g., set 442 and set 444) have the same configuration, and in this embodiment are all inverse-S shaped. Exemplary struts are shown axially disposed relative to the scaffold 406″″, and the scaffold 406″″ may include transition sections which are described elsewhere herein. Scaffold 406″″ may be a proximal scaffold or a distal scaffold, or it may extend along the length of the impeller housing.

The scaffolds and blood conduit embodiments in FIGS. 13A-17 are illustrative, and may be modified to include aspects of other embodiments herein. The following description may provide modifications to the scaffolds in FIGS. 13A-17, any of which may be incorporated into any of the scaffolds in FIGS. 13A-17.

In any of the scaffolds shown in FIGS. 13A-17, at least a first end of each of the plurality of axially extending elements may extend to one or more of a proximal end region (e.g., 417′, 407′) and a distal end region (e.g., 417,) of the expandable scaffold.

In any of the scaffolds shown in FIGS. 13A-17, at least one of, and optionally all of, the plurality of axially extending elements may be linear. In any of the scaffolds shown in FIGS. 13A-17, at least one of, and optionally all of, the plurality of axially extending elements may be curvilinear.

In any of the scaffolds shown in FIGS. 13A-17, each one of the plurality of axially extending elements may have proximal and distal ends, wherein the proximal and distal ends are substantially circumferentially aligned.

In any of the scaffolds shown in FIGS. 13A-17, each of the plurality of axially extending elements may have a circumferential span (illustrated as “CS” in FIG. 15A) that is not larger than 10 degrees circumferentially around the expandable scaffold, optionally not larger than 5 degrees of the expandable scaffold.

In any of the scaffolds shown in FIGS. 13A-17, each of the plurality of axially extending elements may follow a path that is substantially parallel with a longitudinal axis of the expandable scaffold.

In any of the embodiments in FIGS. 13A-17, each of the plurality of axially extending elements may be continuous and axially extending over at least 55% of a length of the expandable scaffold, optionally over at least 60%, optionally over at least 65%, optionally over at least 70%, optionally over at least 75%, optionally over at least 80%, optionally over at least 85%, optionally over at least 90, optionally over at least 95.

In any of the scaffolds shown in FIGS. 13A-17, all of the connectors in all of the sets of the plurality of sets of connectors may have the same configuration. In any of the scaffolds shown in FIGS. 13A-17, all of the connectors in all of the sets of the plurality of sets of connectors may not have the same configuration. In any of the scaffolds shown in FIGS. 13A-17, each individual set of connectors may have a plurality of connectors that have the same configuration. In any of the embodiments in FIGS. 13A-17, all of the connectors in all of the sets of the plurality of sets of connectors may have an S-shape. In any of the embodiments in FIGS. 13A-17, all of the connectors in all of the sets of the plurality of sets of connectors may have a reverse (or inverted) S-shape. In any of the scaffolds shown in FIGS. 13A-17, all of the connectors in a first set of connectors may have a S shape. In any of the scaffolds shown in FIGS. 13A-17, a second set of connectors that is circumferentially adjacent to the first set of connnectors may all have an inverted S shape. In any of the scaffolds shown in FIGS. 13A-17, S shape/inverted S shape connectors may alternate around the circumference of the expandable scaffold.

In any of the embodiments in FIGS. 13A-17, a first set of connectors that extend in a first circumferential direction from a first axially extending element may extend from the fast axially extending element at axial locations that are different from the axial locations at which a second set of connectors extend from the first axially extending element in a second circumferential direction (i.e., the connectors have ends that are axially offset).

In any of the embodiments in FIGS. 13A-17, the expandable scaffold may include a transition region connecting a first axially extending element with a strut, optionally wherein the transition region is considered part of the expandable scaffold. A transition region may also connect the strut with a second axially extending element, the second axially being circumferentially adjacent to the first axially extending around the blood conduit. In any of the scaffolds shown in FIGS. 13A-17, the expandable scaffold may extend along substantially the entire length of the conduit. In any of the scaffolds shown in FIGS. 13A-17, the expandable scaffold may extend along less than 50% of the length of the expandable impeller housing. In any of the embodiments in FIGS. 13A-17, the expandable scaffold may extend only in a region of the expandable housing in which an impeller is disposed.

In any of the embodiments in FIGS. 13A-17, the expandable impeller housing may include a second expandable scaffold axially spaced from the first expandable scaffold. A second expandable scaffold may have an expanded configuration with a second plurality of axially extending elements that are axially extending over at least 50% of a length of the second expandable scaffold and wherein the second expandable scaffold may also include a plurality of sets of connectors, each set of connectors extending circumferentially between first and second circumferentially adjacent axially extending elements. A second expandable scaffold may include any features set forth in any of the claims or described elsewhere herein. In any of the scaffolds shown in FIGS. 13A-17, the expandable scaffold may be unitary, that is, made from a single piece of starting material.

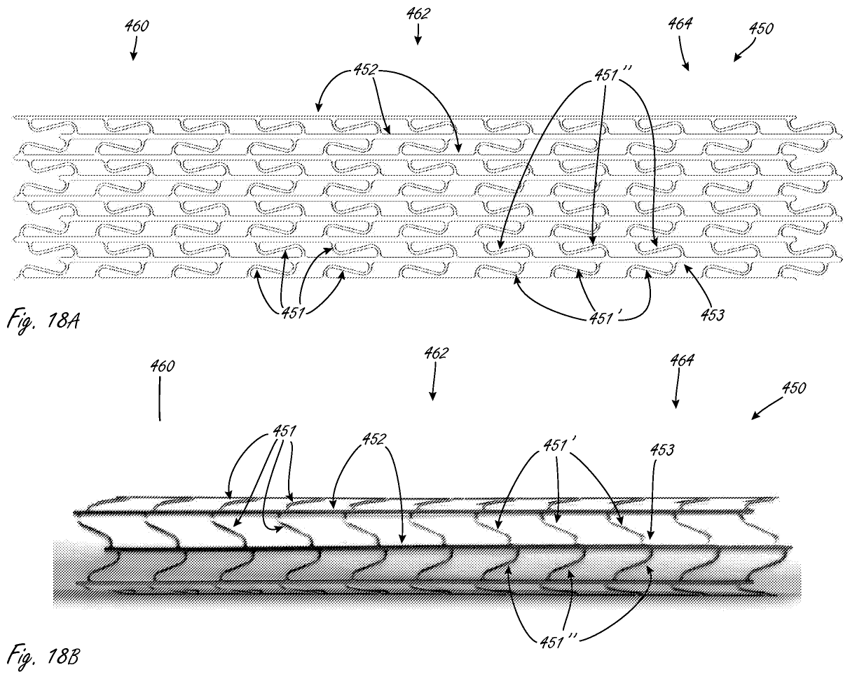

FIGS. 18A and 18B illustrate an exemplary scaffold 450 comprising a plurality of axially extending elements 452 (eight in this example). Scaffold 450 includes a proximal scaffold 460, a central scaffold 462, and distal scaffold 464. In this example axially extending elements 452 are linear. Central scaffold 462 is connected to proximal scaffold 460 and to distal scaffold 464 in this example, and in particular, is unitary with them in this example. FIG. 18B illustrates an expanded configuration, and FIG. 18A illustrates an as-cut flat illustration of the scaffold. The axially extending elements 452 that are labeled in FIG. 18B are circumferentially adjacent axial elements. Adjacent axially extending elements are connected by a plurality of circumferential connectors 451, which in this example have general S or inverse-S configurations, which include at least one bend formed therein. As shown, each circumferential connector is circumferentially adjacent to another circumferential connectors, and together they extend around the blood conduit. In this example, as shown, circumferentially adjacent circumferential connectors are displaced axially relative to one another. For example, circumferential connectors 451′ are axially displaced (or axially offset) relative to circumferential connectors 451″. Axially displaced or axially offset in this context refers to proximal ends of the connectors being axially offset, distal ends of the connectors being axially offset, or both. In this example, a section of each one of the axially extending elements connects adjacent circumferential connectors that are axially displaced. For example, section 453 of one of the axially extending elements 452 connects circumferential connector 451′ and 451″, which creates the axially displaced nature of the circumferentially adjacent circumferential connectors. In this example, distal ends of connectors 451″ are further distally than the distal ends of the circumferentially adjacent connectors 451′, as shown. FIGS. 18A and 18B also illustrate a first group of a plurality of circumferential connectors having a first axial position, and a second group of the plurality of circumferential connectors having a second axial position, wherein the first and second axial positions alternate circumferentially around the blood conduit, as shown.

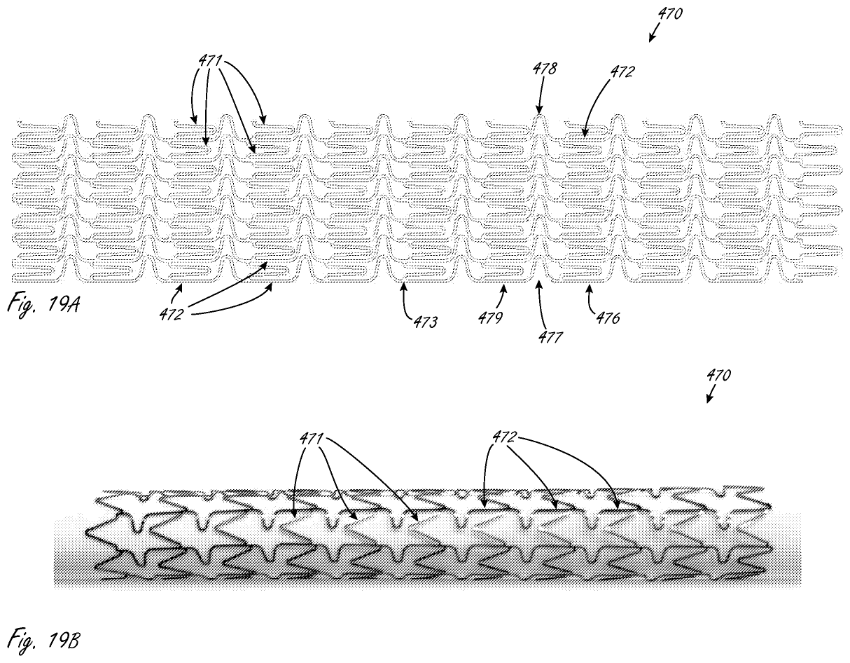

FIGS. 19A and 19B illustrate an exemplary scaffold 470. Scaffold 470 includes a plurality of axially extending elements 472, which are linear is sections but are not linear along the entire scaffold 470 length. Scaffold 470 also includes connectors 471 that circumferentially connect circumferentially adjacent axial elements 472. Connectors 471 includes peaks that are oriented, or point, axially, and in this example may be oriented distally or proximally. Scaffold 470 includes a proximal scaffold, a central scaffold, and a distal scaffold that are connected, and in this example are unitary, just as with the scaffold in FIGS. 18A and 18B. Both the proximal scaffold, central scaffold, and distal scaffold comprise a plurality of linear axially extending elements spaced apart around the blood conduit, wherein first and second adjacent linear axially extending elements are each connected by a circumferential connector having at least one bend formed therein. The circumferential connectors defining a plurality of circumferential connectors around the blood conduit, and wherein circumferentially adjacent circumferential connectors of the plurality of circumferential connectors are displaced axially relative to one another. Like in FIGS. 18A and 19B, a section 473 of each one of the axially extending elements (in this example linear) connects circumferentially adjacent circumferential connectors that are axially displaced, as shown. FIGS. 19A and 19B illustrate a first group of a plurality of circumferential connectors having a first axial position, and wherein a second group of the plurality of circumferential connectors have a second axial position, wherein the first and second axial positions alternate circumferentially around the blood conduit. In this embodiment, the proximal, central, and distal scaffolds generally have the same configuration (except the ends of the distal and proximal scaffolds).

Scaffold 470 also includes second region 477 that is axially adjacent first region 476, wherein second region 477 comprises a plurality of peaks 478 that are shown oriented orthogonally relative to a long axis of the blood conduit (membrane not shown for clarity). In this example, each of the plurality of peaks 478 is an extension of one of the axially extending elements 472 in the first region 476, as shown. Scaffold 470 also includes third region 479 that is axially adjacent second region 477, the third region 470 comprising a second plurality of linear axially extending elements as shown that are spaced apart around the blood conduit, and a second plurality of circumferential connectors 471, where the second region 477 joins the first region 476 and third region 479. In this example this pattern continues along the length of the scaffold.

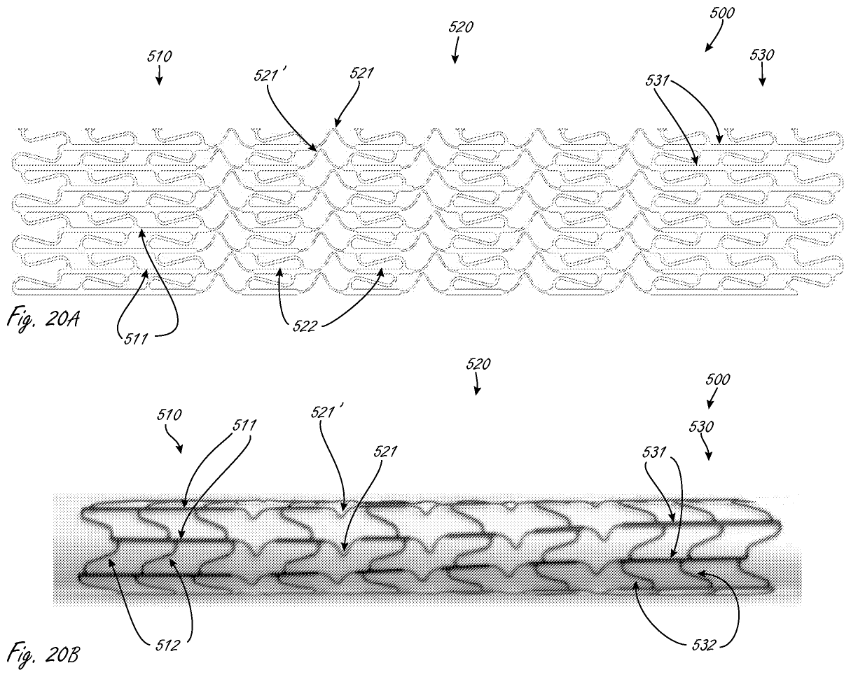

FIGS. 20A and 20B illustrate exemplary scaffold 500, with FIG. 20B showing the expanded configuration and FIG. 20A illustrating a flattened non-expanded configuration. Features that are shown in FIGS. 20A and 20B that are the same as features shown in other scaffolds herein may be expressly included in this embodiment even if not described herewith. Scaffold 500 includes proximal scaffold 510, central scaffold 520 and distal scaffold 530, which are unitary in this embodiment. In this embodiment the central scaffold 520 has a pattern and configuration such that it is less stiff in response to a radially inward force than proximal scaffold 510 and distal scaffold 530. Proximal scaffold 510 may be a proximal impeller scaffold, and distal scaffold 530 may be a distal impeller scaffold, within at least a portion of which a proximal impeller and a distal impeller may be disposed, respectively. Scaffold 500 central scaffold 520 has a pattern that is different than the pattern in scaffold sections 510 and 530. In this example, scaffold sections 510 and 530 have patterns that are substantially the same. Scaffold 500 includes circumferential connectors in proximal scaffold 510, central scaffold 520, and distal scaffold 530, as shown. For example, proximal scaffold 510 includes circumferential connectors 512, and distal scaffold 530 includes circumferential connectors 532. The circumferential connectors in scaffold 500 have the same configurations as circumferential connectors 451 in the scaffold 450 in FIGS. 18A and 18B, and all descriptions thereof are incorporated by reference with the circumferential connectors into all scaffold sections in scaffold 500. For example only, circumferentially adjacent circumferential connectors are axially displaced (i.e., axially offset) relative to one another, which is described in more detail elsewhere herein. The circumferential connectors also have the S and inverse-S configurations, which is described with respect to other scaffolds herein. The central scaffold 520 in scaffold 500 also includes peaks 521 and 521′, similar to peaks 478 in the scaffold in FIGS. 19A and 19B. A first plurality of peaks 521 have a first axial position, and a second plurality of peaks 521′ have a second axial position, which can be seen clearly in FIG. 20A. The axial position alternates circumferentially around the scaffold, as shown. Peaks 521 and 521′ extend from axially extending elements 522 like the scaffold in FIGS. 19A and 19B. The proximal scaffold and the distal scaffold do not include peaks in this embodiment. Axially extending elements 522 in the central scaffold section have a width that is greater than the width of the scaffold in peak 521 regions, as shown. This difference in width can provide the peak regions with greater flexibility, while the wider axially extending element provide sufficient radial support in the central scaffold. Any of the scaffold sections with the peaks may be considered a first region, and the axially adjacent sections with circumferential connectors and axially extending elements may be considered second regions, examples of which are described elsewhere herein. In this embodiment the axially extending elements are linear as shown, but may be curvilinear in other embodiments.

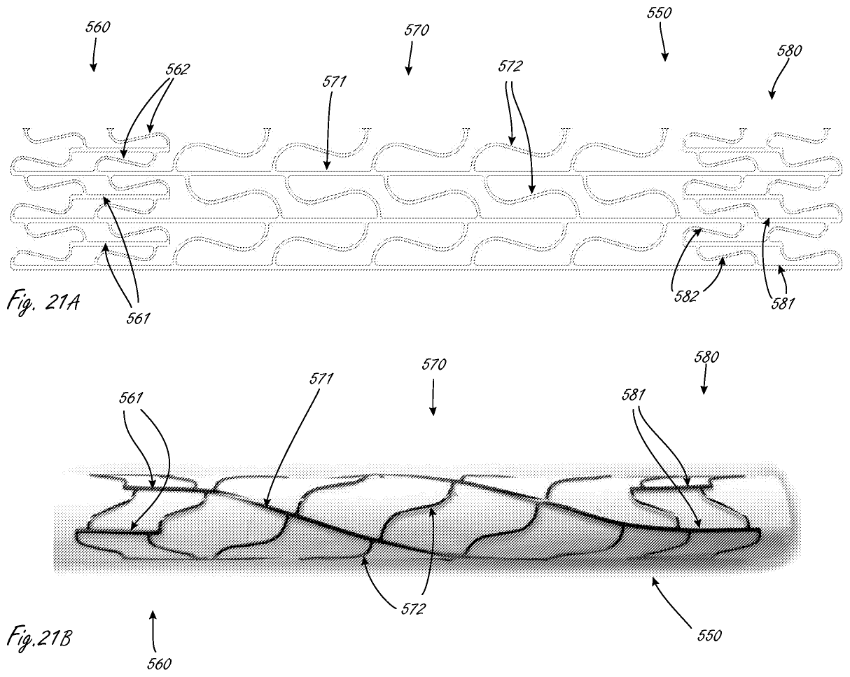

FIGS. 21A and 21B illustrate exemplary scaffold 550, with FIG. 21B showing the expanded configuration and FIG. 21A illustrating a flattened non-expanded configuration. Features that are shown in FIGS. 21A and 21B that are the same as features shown in other scaffolds herein may be expressly included in this embodiment even if not described herewith. Scaffold 550 includes proximal scaffold 560, central scaffold 570 and distal scaffold 580, which are unitary in this embodiment. Proximal scaffold 560 may be a proximal impeller scaffold, and distal scaffold 580 may be a distal impeller scaffold, within at least a portion of which a proximal impeller and a distal impeller may be disposed, respectively. Scaffold 550 central scaffold 570 has a pattern that is different than the pattern in scaffold sections 560 and 580. In this example, scaffold sections 560 and 580 have patterns that are substantially the same. Scaffold 550 includes circumferential connectors in proximal scaffold 560, central scaffold 570, and distal scaffold 580, as shown. For example, proximal scaffold 560 includes circumferential connectors 562, and distal scaffold 580 includes circumferential connectors 582. The circumferential connectors in scaffold 550 have the same configurations as circumferential connectors 451 in the scaffold 450 in FIGS. 18A and 18B, and all descriptions thereof are incorporated by reference with the circumferential connectors into all scaffold sections in scaffold 550. For example only, circumferentially adjacent circumferential connectors are axially displaced (i.e., axially offset) relative to one another, which is described in more detail elsewhere herein. The circumferential connectors also have the S and inverse-S configurations, which is described with respect to other scaffolds herein. Elements 571 in the central scaffold extend into the proximal and distal scaffold sections as shown, forming linear axially extending elements in the proximal and distal scaffolds. Axially extending elements 561 in proximal scaffold 560 do not extend into the central scaffold, as shown. Similarly, axially extending elements 581 in distal scaffold 580 do not extend into the central scaffold, as shown. Elements 571 in the central scaffold 570 have helical configurations as shown. Adjacent elements 571 are connected with connectors 572 as shown. Connectors 572 may have any characteristics of any circumferential connectors herein, such as the alternating S and inverse-S configurations. FIG. 21A illustrates a flattened non-expanded configuration, and the scaffold 550 may be formed into the configuration shown in FIG. 21B, such as by twisting the ends relative to one another and setting the scaffold in the configuration shown in FIG. 21B.

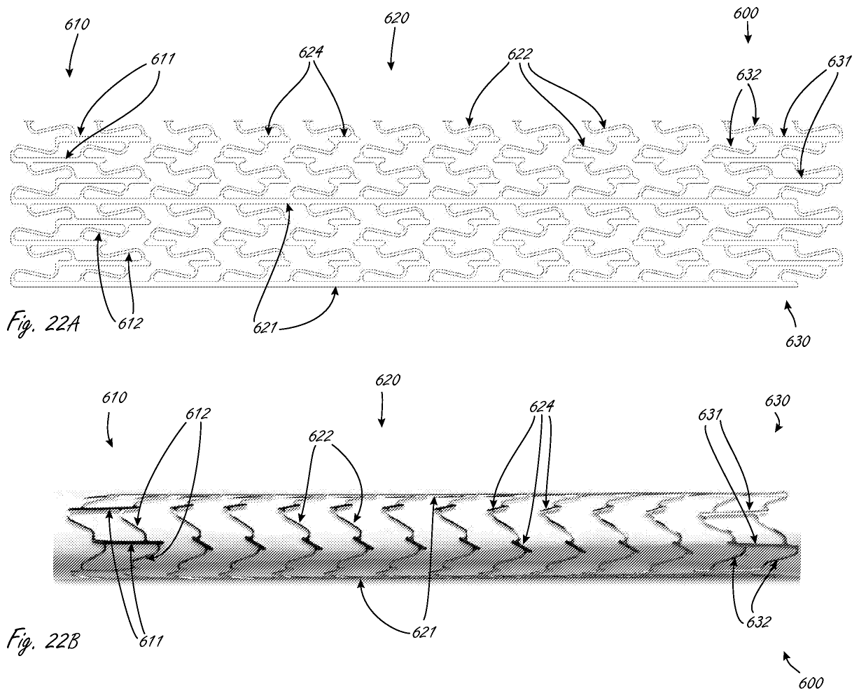

FIGS. 22A and 22B illustrate exemplary scaffold 600, with FIG. 22B showing the expanded configuration and FIG. 22A illustrating a flattened non-expanded configuration. Features that are shown in FIGS. 22A and 22B that are the same as features shown in other scaffolds herein may be expressly included in this embodiment even if not described herewith. Scaffold 600 includes proximal scaffold 610, central scaffold 620 and distal scaffold 630, which are unitary in this embodiment. Proximal scaffold 610 may be a proximal impeller scaffold, and distal scaffold 630 may be a distal impeller scaffold, within at least a portion of which a proximal impeller and a distal impeller may be disposed, respectively. Scaffold 600 central scaffold 620 has a pattern that is different than the pattern in scaffold sections 610 and 630. In this example, scaffold sections 610 and 630 have patterns that are substantially the same. Scaffold 600 includes circumferential connectors in proximal scaffold 610, central scaffold 620, and distal scaffold 630, as shown. For example, proximal scaffold 610 includes circumferential connectors 612, and distal scaffold 630 includes circumferential connectors 632. The circumferential connectors in the proximal and distal sections of scaffold 600 have the same configurations as circumferential connectors 451 in the scaffold 450 in FIGS. 18A and 18B, and all descriptions thereof are incorporated by reference with the circumferential connectors into all scaffold sections in scaffold 600. For example only, circumferentially adjacent circumferential connectors are axially displaced (i.e., axially offset) relative to one another, which is described in more detail elsewhere herein, and connect axially extending elements 611 and 631, respectively. The circumferential connectors also have S and inverse-S configurations, which is described with respect to other scaffolds herein. Axially extending elements 621 in the central scaffold extend into the proximal and distal scaffold sections as shown, wherein the elements 621 are linear axially extending elements in the proximal and distal scaffolds as well as the central scaffold. Axially extending elements 611 in proximal scaffold 610 do not extend into the central scaffold, as shown. Similarly, axially extending elements 631 in distal scaffold 630 do not extend into the central scaffold, as shown. Elements 621 in the central scaffold 620 have axially extending linear configurations as shown. Central scaffold 620 includes axially extending elements 621 that are connected by circumferential connectors. The circumferential connectors include a plurality of axially extending elements 624, each of which connect circumferentially adjacent circumferential connectors 622, as shown. When scaffold 600 is expanded to the configuration shown in FIG. 22B, the circumferential connectors assume the configuration shown, wherein elements 624 are no longer purely axially extending, such that they form an angle with a long axis of the scaffold, as shown.

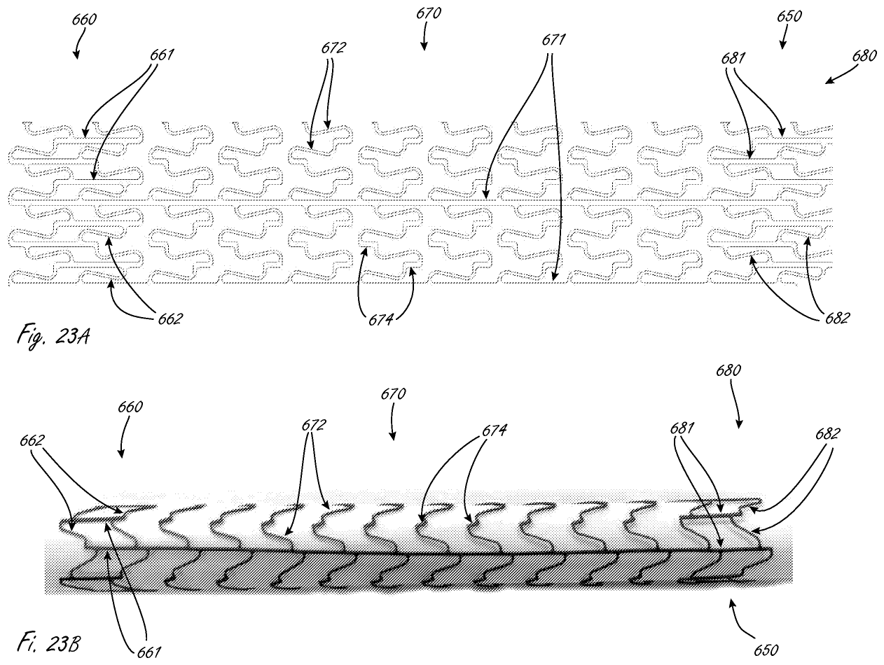

FIGS. 23A and 23B illustrate exemplary scaffold 650, with FIG. 23B showing the expanded configuration and FIG. 23A illustrating a flattened non-expanded configuration. Features that are shown in FIGS. 23A and 23B that are the same as features shown in other scaffolds herein may be expressly included in this embodiment even if not described herewith. Scaffold 650 includes proximal scaffold 660, central scaffold 670 and distal scaffold 650, which are unitary in this embodiment. Proximal scaffold 660 may be a proximal impeller scaffold, and distal scaffold 650 may be a distal impeller scaffold, within at least a portion of which a proximal impeller and a distal impeller may be disposed, respectively. Scaffold 650 central scaffold 670 has a pattern that is different than the pattern in scaffold sections 660 and 680. In this example, scaffold sections 660 and 680 have patterns that are substantially the same. Scaffold 650 includes circumferential connectors in proximal scaffold 660, central scaffold 670, and distal scaffold 680, as shown. For example, proximal scaffold 660 includes circumferential connectors 662, and distal scaffold 650 includes circumferential connectors 682. The circumferential connectors in the proximal and distal sections of scaffold 650 have the same configurations as circumferential connectors 451 in the scaffold 450 in FIGS. 18A and 18B, and all descriptions thereof are incorporated by reference with the circumferential connectors into all scaffold sections in scaffold 650. For example only, circumferentially adjacent circumferential connectors are axially displaced (i.e., axially offset) relative to one another, which is described in more detail elsewhere herein, and connect axially extending elements 661 and 681, respectively. The circumferential connectors also have S and inverse-S configurations, which is described with respect to other scaffolds herein. Axially extending elements 671 in the central scaffold extend into the proximal and distal scaffold sections as shown, wherein the elements 671 are linear axially extending elements in the proximal and distal scaffolds as well as the central scaffold. Axially extending elements 661 in proximal scaffold 660 do not extend into the central scaffold, as shown. Similarly, axially extending elements 681 in distal scaffold 650 do not extend into the central scaffold, as shown. Elements 671 in the central scaffold 670 have axially extending linear configurations as shown. Central scaffold 670 includes axially extending elements 671 that are connected by circumferential connectors. The circumferential connectors include a plurality of axially extending elements 674, each of which connect circumferentially adjacent circumferential connectors 672, as shown. When scaffold 650 is expanded to the configuration shown in FIG. 23B, the circumferential connectors 672 assume the configuration shown, wherein elements 674 are no longer purely axially extending, such that they form an angle with a long axis of the scaffold, as shown. Elements 674 in FIG. 23A are formed by removing material axially disposed between axially adjacent elements 674.

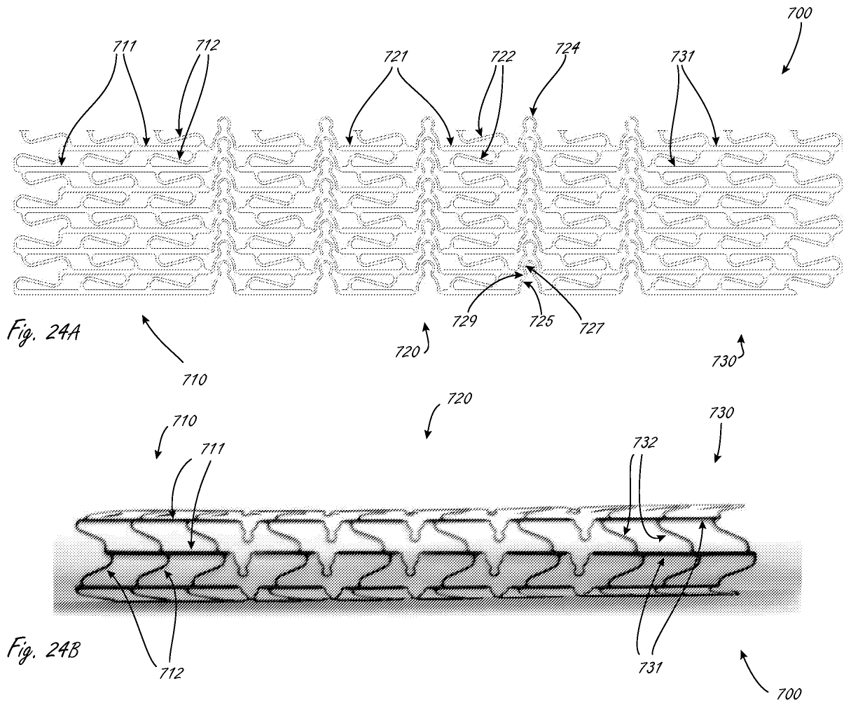

FIGS. 24A and 24B illustrate exemplary scaffold 700, with FIG. 24B showing the expanded configuration and FIG. 24A illustrating a flattened non-expanded configuration. Features that are shown in FIGS. 24A and 24B that are the same as features shown in other scaffolds herein may be expressly included in this embodiment even if not described herewith. For example, scaffold 700 is the same in some ways to the scaffolds shown in FIGS. 19A, 19B, 20A and 20B. Scaffold 700 includes proximal scaffold 710, central scaffold 720 and distal scaffold 730, which are unitary in this embodiment. Proximal scaffold 710 may be a proximal impeller scaffold, and distal scaffold 730 may be a distal impeller scaffold, within at least a portion of which a proximal impeller and a distal impeller may be disposed, respectively. Scaffold 700 central scaffold 720 has a pattern that is different than the pattern in scaffold sections 710 and 730. In this example, scaffold sections 710 and 730 have patterns that are substantially the same. Scaffold 700 includes circumferential connectors in proximal scaffold 710, in central scaffold 720, and in distal scaffold 730, as shown. For example, proximal scaffold 710 includes circumferential connectors 712, and distal scaffold 730 includes circumferential connectors 732. The circumferential connectors in the proximal and distal sections of scaffold 700 have the same configurations as circumferential connectors 451 in the scaffold 450 in FIGS. 18A and 18B, and all descriptions thereof are incorporated by reference with the circumferential connectors into all scaffold sections in scaffold 700. For example only, circumferentially adjacent circumferential connectors are axially displaced (i.e., axially offset) relative to one another, which is described in more detail elsewhere herein, and connect axially extending elements 711 and 731, respectively. The circumferential connectors also have S and inverse-S configurations alternating circumferentially around the scaffold, which is described with respect to other scaffolds herein. Scaffold 700 includes a plurality of axially extending elements 711, which are linear in sections but do not extend along the entire length of scaffold 700. Scaffold 700 also includes circumferential connectors 712 that circumferentially connect circumferentially adjacent axial elements 711. The proximal scaffold, central scaffold, and distal scaffold comprise a plurality of linear axially extending elements 711, 721, and 731, respectively, that are circumferentially spaced apart around the respective scaffold section, wherein first and second adjacent linear axially extending elements are each connected by a circumferential connector 712, 722, and 732, respectively, having at least one bend formed therein. The circumferential connectors define a plurality of circumferential connectors around the scaffold, and wherein circumferentially adjacent circumferential connectors of the plurality of circumferential connectors are displaced axially relative to one another, as shown and described elsewhere herein. As is the case in FIGS. 18A and 19B, a section of each one of the axially extending elements (in this example linear elements) connects circumferentially adjacent circumferential connectors that are axially displaced, as shown. FIGS. 24A and 24B illustrate a first group of a plurality of circumferential connectors having a first axial position, and wherein a second group of the plurality of circumferential connectors have a second axial position, wherein the first and second axial positions alternate circumferentially around the scaffold.

Scaffold 700 also includes a second region that is axially adjacent a first region, wherein the second region comprises a plurality of peaks 724 that are shown oriented orthogonally relative to a long axis of the scaffold 700. In this example, each of the plurality of peaks 724 is an extension of one of the axially extending elements 721, as shown. Scaffold 700 also includes a third region that is axially adjacent the second region, the third region comprising a second plurality of linear axially extending elements as shown that are spaced apart around the scaffold, and a second plurality of circumferential connectors 722, where the second region joins the first region and third region. In this embodiment, the second region includes first convex section 725 and second convex section 727, connected at location 729.

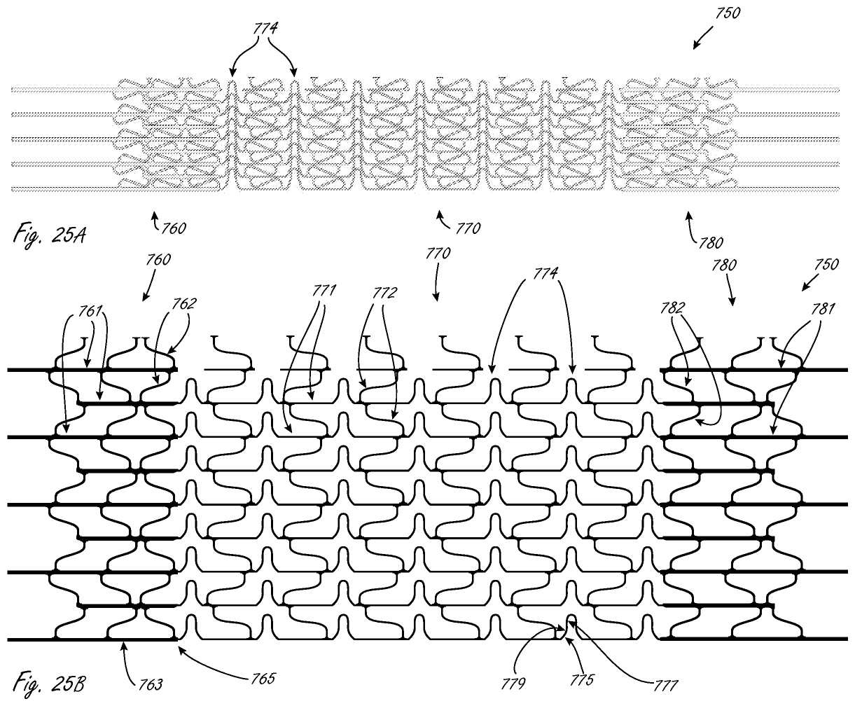

FIGS. 25A and 25B illustrate an exemplary scaffold 750, which in this example includes a proximal scaffold 760, central scaffold 770 and distal scaffold 780, which are unitary. Scaffold 750 is similar in several ways to scaffold 700 in FIGS. 24A and 24B, the disclosure of which is completely incorporated by reference in the description of FIGS. 25A and 25B, any features of which may be included in scaffold 750. One difference is that scaffold 750 central scaffold 770 includes a first region that includes peaks 774, wherein the first region includes sections 775 and 777 connected at location 779, wherein sections 775 and 777 create a smoother curvilinear region than sections 725 and 727 in scaffold 700. An additional difference is that scaffold 750 includes proximal and distal scaffolds that both include mirrored sections, such as sections 763 and 765 as shown in FIG. 25B. The mirrored aspect refers to axially adjacent connectors 762 in section 763 that are mirrored with respect to connectors 762 in section 765. The same mirrored aspect is shown in distal scaffold 780. The mirrored sections in proximal scaffold 760 are closer to central scaffold 770 than the mirrored sections in distal scaffold 780, as shown. In alternative embodiments, mirrored sections in a distal scaffold may be closer to a central scaffold than mirrored sections in a proximal scaffold. The description of all other aspects of scaffolds herein, including axially extending elements and circumferential connectors, are incorporated by reference herein into the scaffold 750. FIG. 25B shows a flat expanded configuration, while FIG. 25A shows a flat non-expanded configuration.

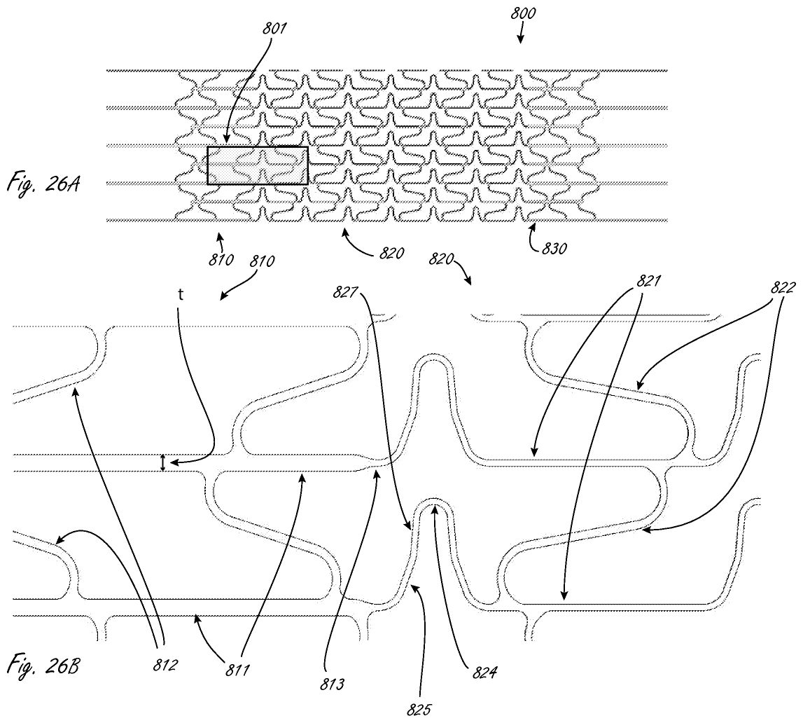

FIGS. 26A and 26B illustrate scaffold 800, which as shown includes many of the same features as scaffold 750 shown in FIGS. 25A and 25B. FIG. 26A illustrate a flattened unexpanded configuration, while FIG. 26B illustrates transition region 801 of scaffold 800 called out in FIG. 26A. A difference between the scaffolds is that in FIGS. 26A and 26B, proximal scaffold 810 includes mirrored sections that are further from central scaffold 820 than mirrored section in distal scaffold, as shown. FIG. 26B illustrates a transition region between proximal scaffold 810 and central scaffold 820. Scaffold 800 includes orthogonally oriented peaks 824 as described elsewhere herein. Scaffold first regions includes sections 825 and 827, which may be the same as sections 775 and 777 in scaffold 750. FIG. 26B illustrates the widths of axially extending elements 811 being greater than the widths of elements 821 in central scaffold, as shown. The thickness measurements are into the page in the figures (in the “z” direction), while the width measurements are in the plane of the page in the figures shown. One thickness “t” of element 811 is labeled for reference. As shown, the thickness “t” of element 811 is greater than the thickness of elements 821 in the central scaffold section.

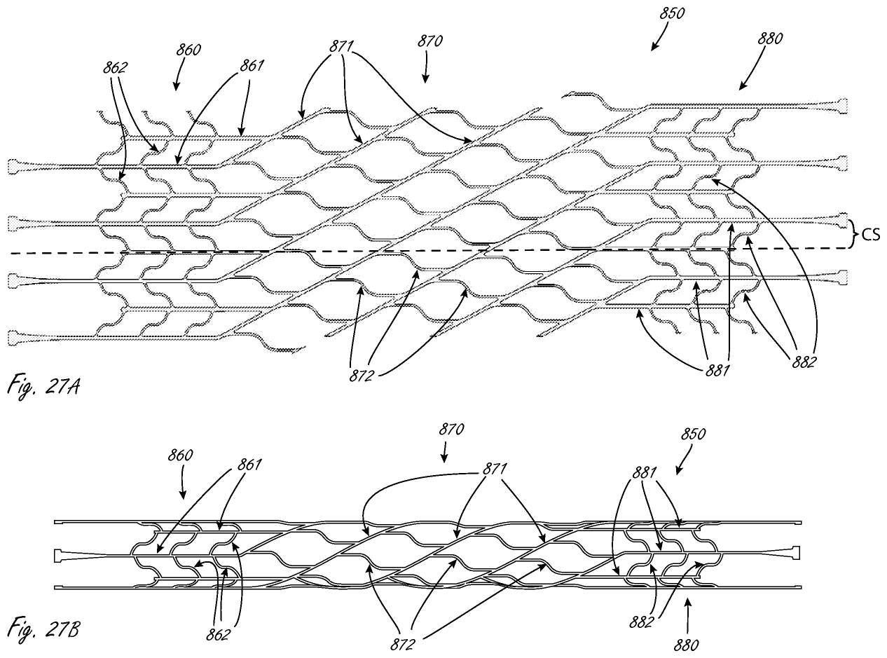

FIGS. 27A and 27B illustrate exemplary scaffold 850, which is similar in several ways to scaffold 550 shown in FIGS. 21A and 21B. Scaffold 850 includes proximal scaffold 860, central scaffold 870 and distal scaffold 880, which in this embodiment may be unitary. Scaffold 850 central scaffold 870 includes helical elements 871 in the non-collapsed configuration (FIG. 27A) and the wrapped configuration (FIG. 27B). In this and any other embodiment herein the scaffold may be manufactured (e.g., including laser cutting of a tubular member) such that the expanded configuration is the configuration is which the scaffold is laser-cut from the tubular member. This is in contrast to any examples herein in which the scaffold is laser cut from a smaller diameter tubular member, and then expanded and set into an expanded configuration. In any of the embodiments herein, a laser cut diameter may be equal to a non-collapsed diameter to, for example without limitation, provide better concentricity. This may also allow coating of a membrane to adhere to struts and have a smoother inner diameter.

Proximal scaffold 860 and distal scaffold 880 have substantial the same configuration, but they are displaced circumferentially by circumferential spacing “CS” (labeled in FIG. 27A). Adjacent helical elements 871 are connected by connectors 872. All other similar aspect of other scaffolds herein may be incorporated herein, including, by way of example only, the axially offset nature of circumferentially adjacent circumferential connectors in proximal scaffold 860 and distal scaffold 880.

FIG. 27A illustrates exemplary distal and proximal struts extending axially from the scaffold, only one strut of which 865 is labeled. In this example there are four proximal and four distal struts. As shown, the struts are tapered and are wider at ends further from the scaffold, which may increase stability over the impellers compared to struts that have a constant width over their entire length. Any of the pump portions herein may include any number of struts that have the same configuration as struts 865.

In any of the embodiments herein, the scaffold may be cut from a tubular member that has an expanded scaffold diameter. In these embodiments, the tubular member has a diameter that is the same or substantially the same as the desired scaffold deployed configuration (un-sheathed). Alternatively, in any of the embodiments herein, the scaffold may be cut from a tubular member that has a non-expanded scaffold diameter. In this embodiments, the tubular member has a diameter less than a scaffold expanded diameter, and after being cut the scaffold may be expanded set in the expanded deployed configuration.

In any of the embodiments herein, a distal scaffold may have a length that is greater than a length of a proximal scaffold. In any of the embodiments herein, a distal scaffold may have a length that is less than a length of a proximal scaffold. In any of the embodiments herein, a distal scaffold may have a length that is the same as a length of a proximal scaffold.

In any embodiment herein, a central scaffold may have a length that is greater than a length of one or both of a proximal scaffold and a distal scaffold.

Any of the different scaffold sections herein may be connected with one or more welds, and may not be unitary with each other.

In any of the embodiments herein, any section or sections of the scaffold may have a thickness (measured radially between a scaffold inner diameter and a scaffold outer diameter) that is the same as or different than a thickness of any other section of the scaffold. For example, a thickness of a scaffold section may be decreased by electropolishing one or more sections more than other sections (which may include no electropolishing). Varying the thickness may be in addition to or alternative to varying the width, which may allow for more design options, as may be desired.

In any of the embodiments herein, an axial distance between proximal and distal scaffold sections may be from 30 mm to 50 mm, such as from 35 mm to 45 mm.

In any of the embodiments herein, the pump portion may be from 40 mm and 80 mm, such as from 50 mm to 70 mm, such as from 55 mm to 65 cm.

In any of the embodiments herein that include first and second impellers, an axial distance between impellers may be from 40 mm to 60 mm, such as from 45 mm to 55 mm.

In any of the embodiments herein, a diameter of the expanded (or non-collapsed) blood conduit may be from 6 mm to 8.5 mm, such as from 6 mm to 8 mm, such as from 6.5 mm to 7.5 mm.

In any of the embodiments herein, a diameter of any of the impellers when expanded may be from 5 mm to 7 mm, such as from 5.5 mm to 6.5 mm.

Some of the pump portions herein include a collapsible and expandable blood conduit, and one or more impellers at least partially disposed in the blood conduit when the pump portion is in an operational state. In some embodiments herein, the collapsible blood conduit includes a scaffold, which may extend along at least a portion of the length of the blood conduit and provide radial support to the blood conduit. In some embodiments herein a scaffold may be unitary along the blood conduit. In some embodiments different scaffold sections may not be unitary (formed from the same starting material), but they may be directly attached or connected to each other (e.g., welded directly together).

The disclosure also includes catheter blood pumps that include one or more sensors thereon or therein, their methods of manufacture, and use. For example only, any blood pumps herein may include one or more sensors configured to sense pressure. A sensor configured to sense blood pressure may be included on an intravascular blood pump for a variety of purposes, such as, for example without limitation, estimating flow or detecting the position of the blood pump. Additionally, for example, one or more sensors may be axially spaced apart (e.g., one near an inflow and one near an outflow) and used to determine a differential pressure across the pump portion.

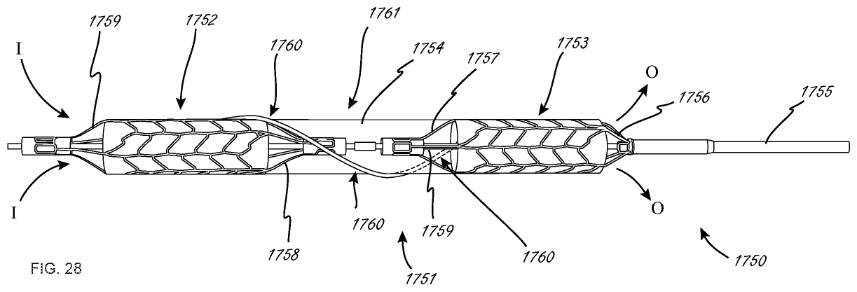

FIG. 28 illustrates an exemplary catheter blood pump 1750 including an expandable and collapsible pump portion 1751 (shown expanded or deployed) disposed distally relative to an elongate body 1755, the pump portion including an expandable impeller housing 1761 that includes a blood conduit that defines a blood lumen between an inflow “I” and an outflow “O”. The pump portion includes one more impellers, any of which may at least partially be disposed axially within the fluid lumen (impellers are not shown in FIG. 28 for clarity). Expandable impeller housing 1761 includes a sensor wire housing 1760 extending at least partially along a length of the expandable impeller housing. Pump portion 1751 also includes a sensor wire (e.g., a fiber optic) secured to a sensor, with the sensor wire housing secured relative to the expandable impeller housing. The sensor wire is disposed within the sensor wire housing 1760, and the sensor wire may be sized such that it floats within a sensor wire lumen defined by the sensor wire housing. As used herein, a sensor wire housing generally defines a sensor wire lumen, in which a sensor wire may be disposed. This disclosure may, however, use the phrases sensor wire lumen and sensor wire housing interchangeably, however, the lumen is generally considered the space within a structural housing. Expandable impeller housings herein may also be referred to as expandable housings herein.

In the embodiment in FIG. 28, sensor wire housing 1760 (which defines a lumen therein) has a helical configuration along at least a portion of the expandable housing 1761, and it may have a helical configuration along as at least 50% of a length of the expandable housing, 55%, 60%, 65%, 70%, 75%, 80%, 85%, 90%, 95%, or 100% of a length of the expandable housing.

The sensor wire housings herein may have a linear configuration along at least a portion of the expandable housing, such as at least 50% of a length of the expandable housing, 55%, 60%, 65%, 70%, 75%, 80%, 85%, 90%, 95%, or 100% of a length of the expandable housing.

The sensor wire housings herein may have a helical configuration along a portion of its length, and may have linear or other configurations along other portions of its length. The sensor wire housings herein may have helical configurations in one or more discrete axially spaced helical regions, and optionally may have linear configurations in one or more discrete axially spaced linear regions. Sensor wire housings may have other non-linear and non-helical configurations as well.

The sensor wire housings herein generally help protect the one or more sensor wires (e.g., fiber optic). Sensors wires (e.g., fiber optics) may be quite fragile and susceptible to breaking, especially when the pump portion is navigated through curved vasculature and bends. Sensor wire housings herein can be sized relative to the sensor wire such that the sensor wire may float within the lumen, which may provide space for the wire to move slightly while the pump portion is navigated and/or in use, which may reduce the likelihood of sensor wire breakage.

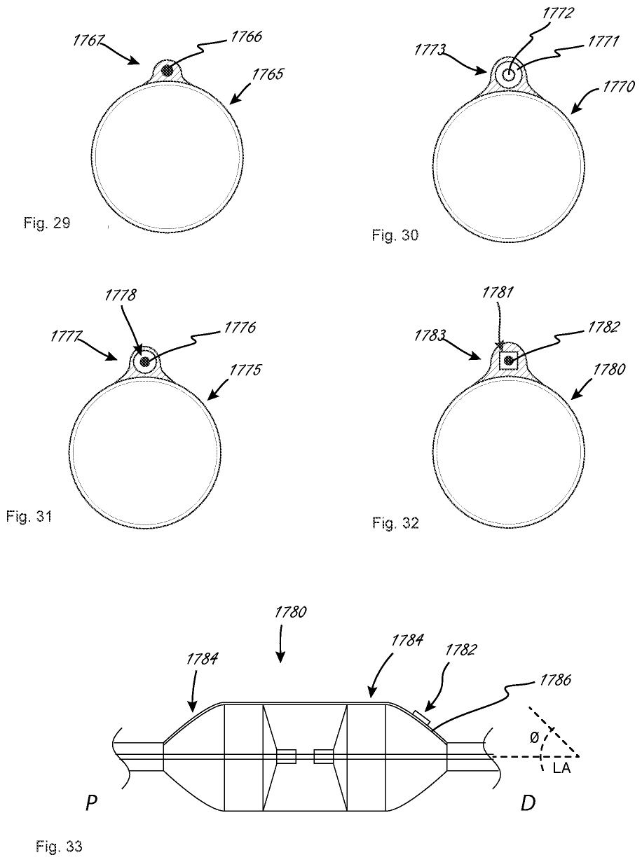

In some embodiments, however, a sensor wire may be fixed relative to an impeller housing such that it is not floating with a space. When described as being fixed relative to an impeller housing, there may be some degree of slight movement provided between a sensor wire and impeller housing due to the flexibility of the materials, but fixed in this context refers generally to not freely floating within an open lumen. FIG. 29 provides an illustrative cross section of expandable housing 1765 (details of which are not shown for clarity, but may include any features of any pump portion herein, such as a membrane, an expandable support member, and impeller, etc., exemplary details of which can be found elsewhere herein), with sensor wire 1766 fixed relative thereto (not floating), and secured thereto by overlay 1767, which may be deposited on the sensor wire to secure wire 1766 relative to housing 1765. The overlay 1767 and sensor wire 1767 may have any configuration along the length of the expandable housing, such as helical, partial helical, curvilinear, partial curvilinear, linear, partially linear, or any combination thereof.

FIG. 30 illustrates an exemplary cross section of exemplary expandable impeller housing 1770 (again, details of which are not shown for clarity, but may include any feature of any pump portion herein, such as a membrane, an expandable support member, and impeller, etc., exemplary details of which can be found elsewhere herein). In this embodiment, the pump portion includes a sensor wire housing that defines a sensor wire lumen that is sized and configured relative to the sensor wire such that the sensor wire floats within the lumen along at least a portion of the expandable impeller housing. In any of the embodiments that include a sensor wire housing, the sensor wire may be fixed to the expandable housing at one more discrete locations, such as at locations where the sensor wire extends out of the sensor wire housing, such as at one or both of a proximal end or a distal end of a sensor wire housing. In the embodiment in FIG. 30, the pump portion includes a separate sensor wire housing that defines a sensor wire lumen 1771. For example only, the sensor wire housing may be a hollow tubular element that extends along at least a portion of the expandable housing, such as a tube. The sensor wire housings herein, in the context of sensor wire lumens, may be a wide variety of materials, such as elastomeric or semi-rigid, or rigid. In any of the embodiments herein, the sensor wire housing may not impart a meaningful increase in rigidity to the expandable impeller housing at the location of the sensor wire housing, although there may be a slight increase in stiffness.

Any of the sensor wire housings herein that house a sensor wire may also have a non-circular cross sectional shape, such as rectilinear (e.g., triangular, rectangular, square), or curvilinear (e.g., oval), or any other non-defined, irregular, shape. In this exemplary embodiment, the sensor wire housing that defines lumen 1771 is secured to the expandable housing 1770 at least partially by overlay 1773, and in this embodiment overlay 1773 is disposed about a radially outermost portion of the sensor wire housing and lumen 1771. The overlay 1773 at least partially serves to help secure the sensor wire housing relative to the expandable housing. In this exemplary embodiment it may be a combination of the expandable membrane material of the housing 1770 as well as overlay 1773 that together surround the sensor wire housing and help secure it relative to the expandable housing 1770. The membrane of the expandable impeller housing 1770 is disposed radially within sensor wire housing, and overlay 1773 is disposed about the sensor wire housing and lumen 1771, including about a radially outmost portion of the sensor wire housing as shown. In any of the embodiments herein, the expandable housing 1770 membrane may not be in direct contact with the sensor wire housing; there may be one or more layers of overlay material in between the two.

Any of the overlays herein may be different than the expandable housing membrane in one or more ways. For example, possible differences herein in this context include, for example, one or more of chemical structure, durometer, stiffness, and thickness. For example, an overlay is considered different than a conduit membrane in this context if the overlay is the same material as a membrane, but has a different durometer. Additionally, for example, an overlay is considered different than an impeller housing membrane in this context if the overlay is the same material as a membrane, but has a different thickness than the membrane.

In any of the embodiments herein, an overlay may comprise a polymeric material, optionally a urethane, and optionally polycarbonate based. In any of the embodiments herein, a membrane that at least partially defines a blood flow lumen may comprise a polymeric material, optionally a urethane, and optionally polycarbonate based. In any of the embodiments herein, the membrane may have the same chemical structure as the overlay.

FIG. 31 illustrates an exemplary embodiment in which a sensor wire lumen 1778 is not defined by a separate structural sensor wire housing, such as in the embodiment of FIG. 30. In the example of FIG. 31, lumen 1778 is defined by a combination of overlay 1777 and the expandable housing 1775. By way of example only, the sensor wire lumen in FIG. 31 may be created by creating a pump portion as shown in FIG. 30 (whether the sensor wire 1772 has been positioned as shown or not), and then removing the sensor wire housing to thereby create lumen 1778 now defined by overlay 1777 and the expandable housing 1775. In some embodiments the overlay may comprise one or more polymeric materials, and the wire lumen may be defined by one or more polymeric materials. Expandable housing 1775 may, again, include any feature of any expandable housing herein, such as a membrane, an expandable support member, and impeller, etc., exemplary details of which can be found elsewhere herein. Sensor wire 1776 is shown floating in lumen 1778.

FIG. 32 illustrates an exemplary cross section of an embodiment of an expandable housing 1780 (again, impeller not shown for clarity) that includes sensor wire 1782 floating within lumen 1781, wherein lumen 1781 has a non-circular cross section. In this embodiment, the cross section is rectilinear (e.g., rectangular, square). The cross section can be created by first positioning a rectilinear structure element over the expandable housing 1780, then removing it after overlay 1783 has been deposited on top of it, similar to the description of FIG. 31. Lumen 1781 may be also defined by a sensor wire housing structural member that is secured with overlay 1783.

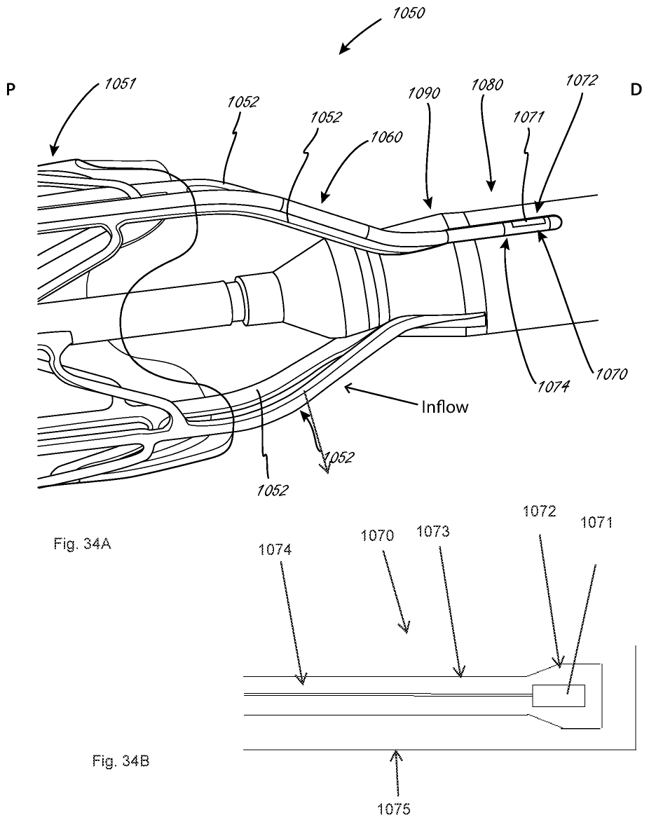

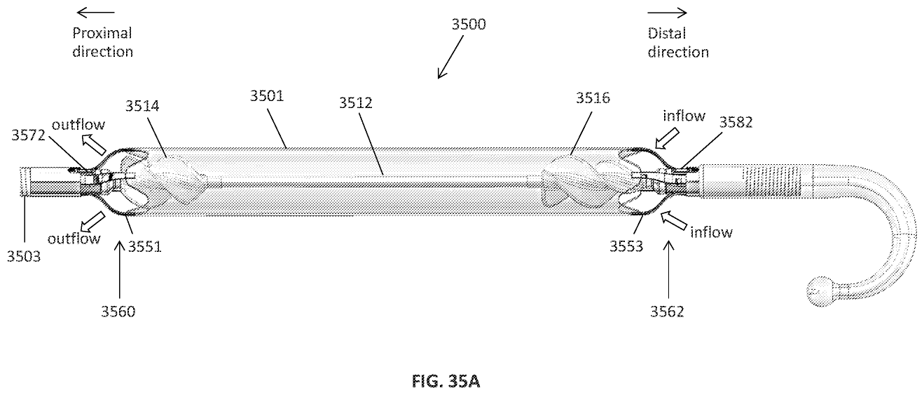

FIG. 33 is a side view of a pump portion that includes an exemplary expandable impeller housing 1780 that includes sensor 1782 coupled to the expandable housing 1780, and sensor wire lumen 1784 (and a sensor wire therein) extending in a linear configuration along the expandable housing. Sensor wire lumen 1784 may be any of the wire lumens herein. Expandable impeller housing 1780 may be any of the expandable housings herein, including any that include more the one impeller, and any that include one or more expandable support members that help provide structural support to the expandable housing.

In FIG. 33, sensor 1782 (which may be a pressure sensor) is secured to a distal strut 1786 of the expandable housing, wherein the strut is near the inflow of the pump portion. Strut 1786 may be any of the struts described herein or in any reference incorporated herein by reference. The sensors herein may be directly or indirectly secured to one or more expandable portion reinforcing elements (e.g., a strut, or an element of a scaffold). In this embodiment, the sensor is secured to an element (e.g., a strut) extending radially inward relative to a portion of the expandable housing at least partially surrounding an impeller. Any of the sensors herein can be coupled to an element with this configuration.

Any of the embodiments described herein can include at least one sensor on an inflow of the blood pump (configured for measuring ventricular pressure) and at least one sensor on an outflow of the blood pump (configured for measuring aortic pressure).

In this embodiment (and any embodiment herein), the sensor may be secured such that a pressure sensitive area of the sensor is not orthogonal to a longitudinal axis of the expandable housing, and is optionally between 1 and 89 degrees relative to the longitudinal axis, such as from 5-85 degrees, such as from 10-80 degrees. The reference angle theta is shows in FIG. 33.

In any of the embodiments herein, the sensor wire extends along the expandable housing and is in communication with a proximal region of the blood pump that is spaced to remain outside of a patient when the impeller is in use. Information sensed from the one or more sensors can be used for one or more of the following: estimating flow, and detecting the position of the blood pump. Additionally, one or more sensors may be axially spaced apart (e.g., one near an inflow and one near an outflow, not shown), and used to determine a differential pressure across the pump portion.

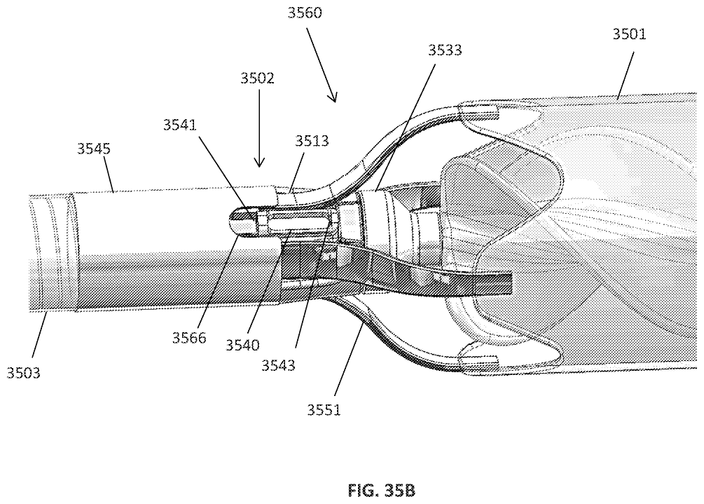

FIG. 34A illustrates a distal region of an exemplary pump portion 1050 showing collapsible blood conduit 1051 in an expanded configuration. The inflow to the blood conduit is shown, and an optional distal impeller is not shown. However it should be understood that other embodiments will not include a distal impeller, but will only include a proximal impeller. The blood conduit 1051 may comprise any aspect of any of the expandable and collapsible blood conduits herein, including for example, any of one or more scaffolds, any of the one or more baskets, and any of the one or more membranes secured thereto. Pump portion 1050 includes distal struts 1052, in this example four, but more or fewer may be included. Distal struts 1052 may be unitary or connected with a scaffold of the blood conduit 1051, and are shown extending distally therefrom and radially inward towards a distal hub or radially central region, which may include a distal bearing housing 1090 and one or more other components 1080 that are included in a distal end region of the pump portion.

In this example, and which is similar to embodiments above that include a distal sensor, sensor components are secured relative to one of the distral struts 1052. Pump portion 1050 includes a sensor connection housing 1060 extending along the blood conduit and along one of the distal struts 1052 as shown. Sensor connection housing 1060 may be any sensor connection housing herein that may house therein one or more sensor connections (e.g., wires) that are coupled to a distal sensor 1072 and communicate information sensed therewith. The one or more sensor connections may extend all the way proximally through the pump catheter to an external console that is adapted to receive signals communicated along the sensor connections, such as signals indicative of pressure sensed from the pressure sensor. Sensor connection housing 1060 in this embodiment extends distal to the one or more impellers, and continues to a location 1074 where it meets sensor housing 1070. Sensor housing 1070 is sized and configured to receive sensor 1072 at least partially therein. Sensor 1072 may be, for example, a pressure sensor.

Sensing housing or carrier 1070 is secured distal to the one or more impellers, and is secured to the distal end region of the pump portion, such as to a hub or other centrally located component 1080. In this example, distal struts 1052 have distal ends that are coupled to the distal end region, such as to distal end hub component 1080.

In this example, as shown, sensor 1072 has a pressure sensitive face 1071 that faces radially outward, in a direction orthogonal to a long axis of the pump portion 1050, such that it is facing blood flowing past sensor 1072 toward the inflow of blood conduit 1051.

Sensor housing 1070 may have a channel or depressed region formed therein that is sized and configured to receive sensor 1072 therein and optionally any distal regions of connectors (e.g., wires) that are coupled to and in communication with sensor 1072, such as any of the wires herein extending along the blood conduit. An encapsulating material such as silicone may be deposited at least partially about the sensor 1072 and into the channel or depression in the housing to encapsulate the sensor 1072 relative to housing 1072 and help secure sensor 1072 to housing 1070. Housing 1070 also functional acts as a base to help stabilize the sensor relative to the pump. The central component 1080 is considered to be an axially extending component with a radially outward surface, and housing 1070 may be secured relative thereto so that sensor 1072 faces outward as is shown. A pressure sensor facing outward as shown and secured near the outflow to an axially extending component may provide more accurate pressure sensor readings near the inflow.

FIG. 34B illustrates an exemplary top view of a distal region of sensor housing or carrier 1070 that may be incorporated into the pump design of FIG. 34A. Carrier 1070 includes a channel or recessed depression 1073 formed in a main housing body 1075. Sensor 1072 and associated connectors one or more connectors 1074 (e.g., wires) are shown diposed within the channel or recessed region 1073 therein. As set forth above, an encapsulating material may be disposed about the sensor 1072 and/or associated one or more connectors 1074, such as in the volume between the sensor 1072 and the channel or recessed depresion 1073 formed in body 1075 to help stabilize the sensor relative to the body portion of the housing.

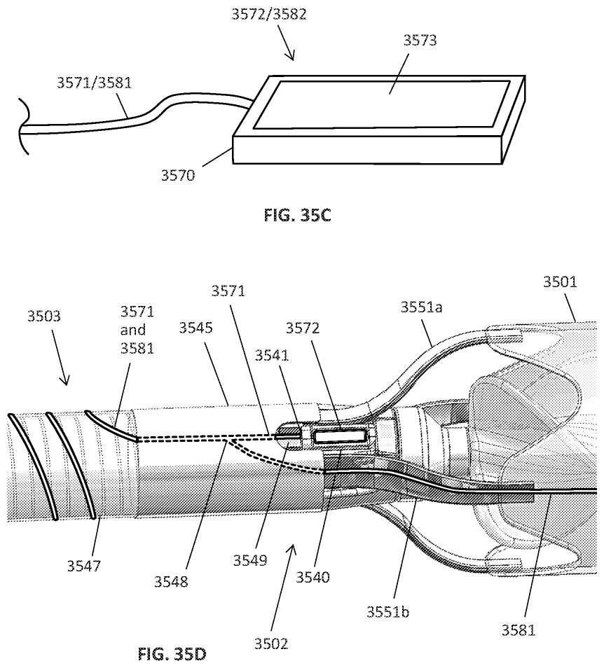

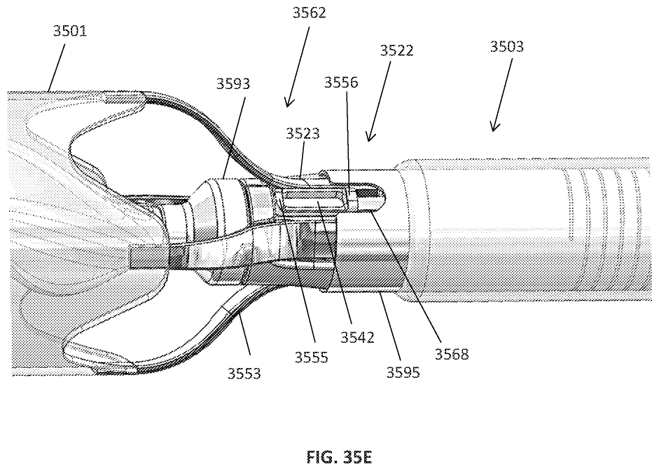

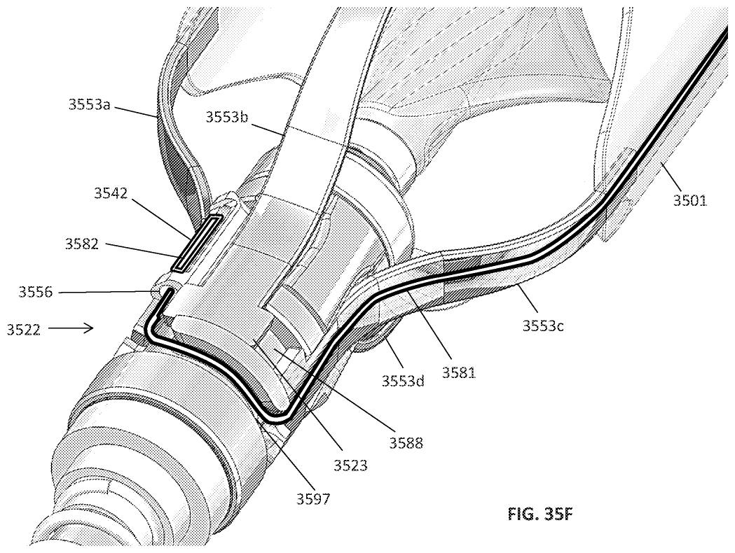

FIGS. 35A-35F illustrate an exemplary catheter blood pump 3500 having another sensor arrangement. FIG. 35A illustrates a side view of the exemplary catheter blood pump 3500. The catheter blood pump 3500 may be coupled to a distal end of a catheter shaft 3503. The catheter blood pump 3500 may include a blood conduit 3501, which may be a tubular structure that defines a channel for blood to flow through. The blood conduit 3501 may include any aspect of the blood conduits described herein, including for example, any of one or more scaffolds, any of the one or more baskets, and any of the one or more membranes. The blood conduit 3501 may be expandable and collapsible. When expanded, the blood conduit 3501 may define an inner lumen configured to allow blood to flow therethrough. An outer sheath/shaft (see e.g., catheter sheath 322, FIG. 12C) may be configured to cover the catheter blood pump 3500 when the catheter blood pump 3500 is in a collapsed state. For example, the outer sheath/shaft may be pushed in the distal direction to cover the catheter blood pump 3500 and pulled in the proximal direction to uncover the catheter blood pump 3500.

A proximal end of the blood conduit 3501 may include proximal struts 3551 that extend from the proximal end of the blood conduit 3501 and secure the proximal end of the blood conduit 3501 to a proximal radially central region (e.g., a proximal hub) of the catheter blood pump 3500. The proximal struts 3551 may exend from the blood conduit 3501 in a proximal direction relative to the blood conduit 3501. A distal end of the blood conduit 3501 may include distal struts 3553 that extend from the distal end of the blood conduit 3501 and secure the distal end of the blood conduit 3501 to a distal radially central region (e.g., the distal hub) of the catheter blood pump 3500. The distal struts 3553 may extend from the blood conduit 3501 in a distal direction relative to the blood conduit 3501.

The proximal struts 3551 and distal struts 3553 may be configured to collapse radially inward to position the blood conduit 3501 in a collapsed state and to expand radially outward to position the blood conduit 3501 in an expanded state. When the blood conduit 3501 is in an expanded state, as shown in FIG. 35A, the proximal struts 3551 and distal struts 3553 may be sufficiently spaced apart to allow blood to flow therebetween to enter and exit the blood conduit 3501 through proximal and distal openings of the blood conduit 3501. In the example shown, the pump portion 3500 includes four proximal struts 3551 and four distal struts 3553; however, more or fewer proximal struts 3551 and/or distal struts 3553 may be included.

A proximal impeller 3514 and a distal impeller 3516 may be at least partially disposed within the blood conduit 3501. The proximal impeller 3514 and the distal impeller 3516 may be in operable communication with a drive cable 3512. The proximal impeller 3514 and a distal impeller 3516 may be configured to rotate to move blood through the blood conduit 3501. In some examples, the proximal impeller 3514 and distal impeller 3516 may be configured to move blood into the blood conduit 3501 at the distal opening of the blood conduit 3501, through the blood conduit 3501, and out the proximal opening of the blood conduit 3501, as indicated by inflow/outflow arrows in FIG. 35A. The proximal impeller 3514 and distal impeller 3516 may be configured to radially collapse and expand. In an expanded state (as shown in FIG. 35A), the proximal impeller 3514 and distal impeller 3516 may be rotatable to cause blood flow. Collapsing the proximal impeller 3514 and distal impeller 3516 may reduce the diameter of the pump portion 3500 for transporting the pump portion 3500 through the patient's vessels and/or heart. It should also be understood that, while the embodiment of FIG. 35A shows two impellers, in other embodiments this blood pump can include only a single impeller, such as only a proximal impeller or only a distal impeller.

One or more proximal sensor transducers 3572 (e.g., one or more proximal pressure transducers) may be configured to sense one or more inputs at a proximal region 3560 of the blood pump 3500. One or more distal sensor transducers 3582 (e.g., one or more distal pressure transducers) may be configured to sense one or more inputs at a distal region 3562 (e.g., inflow region) of the blood pump 3500. The signals from the sensor transducers 3572 and 3582 may be proximally transmitted through connectors (e.g., wires) along a length of the catheter shaft 3503 to one or more controllers, for example, to a computer console associated with the catheter blood pump 3500. The proximal and distal sensor transducers 3572 and 3582 may be used to during positioning of the pump portion 3500 within the patient's body and/or to determine whether the catheter blood pump 3500 is working properly. In the case of pressure sensors, the transducers 3572 and 3582 may be used to determine a differential pressure across the catheter blood pump 3500.

FIG. 35B shows a closeup view of the proximal region 3560 (e.g., outflow region) of the catheter blood pump 3500. The proximal region 3560 includes a proximal hub 3502 that the proximal ends 3513 of the struts 3551 are coupled to. The proximal hub 3502 may refer to region of the catheter blood pump 3500 that is axially proximally located with respect to the blood conduit 3501. The proximal hub 3502 may have an outer surface that is radially inward with respect to the blood conduit 3501 when in an expanded state. In some cases, the proximal hub 3502 houses a proximal bearing assembly that may include one or more bearings (e.g., 1, 2, 3, 4, 5, etc.) associated with rotational movement of the drive cable 3512 (FIG. 35A). In some cases, a tapered bearing cap 3533 covers a portion of a distal side of the proximal hub 3502.

The proximal hub 3502 may include a proximal sensor housing 3540, which may be configured to secure a sensor transducer (e.g., pressure transducer) therein. The proximal sensor housing 3540 may axially overlap with and/or be radially exterior to the bearing assembly within the proximal hub 3502. As shown, the proximal sensor housing 3540 may be located in the proximal hub 3502 in between the struts 3551, and more specifically at the location of where the proximal ends 3513 of the struts attach to the proximal hub. The proximal sensor housing 3540 may include a recess that is shaped and sized to accommodate the sensor therein. The sensor transducer may be mechanically fixed in proximal sensor housing 3540 any of a number ways. For example, one or more adhesives and/or potting materials may be used to secure the sensor transducer within the proximal sensor housing 3540. In some cases, the sensor transducer may alternatively or additionally be secured within the proximal sensor housing 3540 by one or more mechanical features, such as a press-fit feature or undercut lip of the proximal sensor housing 3540. The proximal sensor housing 3540 may include one or more openings for one or more wires to extend out of the proximal sensor housing 3540. In the example shown, the housing 3540 incudes a proximal opening 3541 and a distal opening 3543.

As shown, the ends 3513 (also referred to as “strut feet”) of the struts 3551 may be coupled to the proximal hub 3502. A proximal sleeve 3545 may at least partially cover the strut feet 3513. This arrangement may provide a radially inward force to retain/secure the struts 3551 to the proximal hub 3502. In some cases, the proximal sleeve 3545 may include a cutout 3566 to accommodate the proximal sensor housing 3540. The proximal sleeve 3545 may be coupled to the proximal hub 3502 any of a number of ways. For example, the proximal sleeve 3545 may be bonded (e.g., welded, molded, bonded using an adhesive) to the proximal hub 3502. In some cases, the proximal sleeve 3545 may be press-fit with the proximal hub 3502. In other examples, the proximal sleeve 3545 may be free-floating with respect to the proximal hub 3502.

FIG. 35C illustrates a closeup view of an exemplary pressure sensor transducer 3572/3582. The pressure transducer 3572/3582 may include a sensor body 3570 that encloses electronic circuitry and supports an outward facing pressure sensitive face 3573. The pressure sensitive face 3573 may be flexible and responsive to pressure applied thereon. The pressure sensitive face 3573 may be configured to measure fluid pressure applied thereon. The sensor body 3570 may be secured to the catheter blood pump 3500 such that the pressure sensitive face 3573 is positioned outward and exposed to blood flow entering or exiting the blood conduit 3501. In some cases, the pressure sensitive face 3573 may be configured to measure static pressure in the inflow and/or outflow regions of the blood conduit. One or more wires 3571/3581 may electrically communicate signals associated with sensed pressure from the pressure sensor transducer 3572/3582 to one or more controllers. Additionally or alternatively, the one or more wires 3571/3581 may be configured to electrically communicate signals from the one or more controllers to the pressure transducer 3572/3582. The one or more wires 3571/3581 may include one or more layers of insulative covering.

FIG. 35D illustrates a closeup view of the proximal region 3560 having an exemplary proximal sensor transducer 3572 housed within the proximal sensor housing 3540. A first wire 3571 that provides electrical communication for the proximal sensor transducer 3572 may pass through the proximal opening 3541 of the proximal sensor housing 3540 in the proximal direction toward the catheter shaft 3503. This arrangement may allow the wire 3571 to be directed toward the catheter shaft 3503 with minimal bending of the wire 3571. In some cases, at least part of the first wire 3571 may be mechanically coupled to at least part of a second wire 3581 that provides electrical communication for the distal sensor transducer (3582 in FIG. 35A). The first and second wires 3571 and 3581 may be coupled at a wire junction region 3548 such that the first and second wires 3571 and 3581 travel together in a proximal direction along the catheter shaft 3503. In other cases, the first wire 3571 and the second wire 3581 are not mechanically coupled together and the first and second wires 3571 and 3581 travel independently in the proximal direction along the catheter shaft 3503.

The first and second wires 3571 and 3581 may traverse along the catheter shaft 3503 in any of a number of ways. In the example shown in FIG. 35D, at least a portion of the first and second wires 3571 and 3581 wind helically around the catheter shaft 3503. The first and second wires 3571 and 3581 may wind in a clockwise direction or a counterclockwise direction around the catheter shaft 3503. The pitch (spacing) of the winding may be chosen to minimize crimping/bending of the first and second wires 3571 and 3581 and to allow articulation of the catheter shaft 3503. In some cases, the pitch may vary along the catheter shaft 3503, for example, based on which portions of the catheter shaft 3503 are expected to bend and/or to the degree of bending. In some cases, at least a portion of the first and second wires 3571 and 3581 may travel in a linear (non-winding) path along the catheter shaft 3503. In cases where the first and second wires 3571 and 3581 are not coupled together and travel independently along the catheter shaft 3503, one or both of the first wire 3571 and the second wire 3581 may wind in a clockwise direction or a counterclockwise direction. In some cases, one or both of the first and second wires 3571 and 3581 travel in a linear (non-winding) path along the catheter shaft 3503. In some examples, part of the first wire 3571 and/or the second wire 3581 wind around the catheter shaft 3503 while another part of the first wire 3571 and/or the second wire 3581 travel in a linear path along the catheter shaft 3503.

In the example shown in FIG. 35D, the first and second wires 3571 and 3581 are positioned under the proximal sleeve 3545 such that the proximal sleeve 3545 covers the first and second wires 3571 and 3581. In other examples, the first wire 3571 and/or the second wire 3581 is/are positioned over the proximal sleeve 3545. In some cases where the first and second wires 3571 and 3581 are coupled together at a wire junction region 3548, the wire junction region 3548 may be axially aligned with the proximal sleeve 3545 (e.g., be positioned below the proximal sleeve 3545). In other cases, the wire junction region 3548 may be positioned proximal or distal to the proximal sleeve 3545.

In some cases, the first and second wires 3571 and 3581 may be secured to the proximal hub 3545 and/or the catheter shaft 3503 using one or more securing means. In some examples, the first and second wires 3571 and 3581 may be bonded to the proximal hub 3545 and/or the catheter shaft 3503.

Such bonding may include welding, molding, or adhearing using one or more adhesives. In some examples, a laminate material (e.g., shrink wrap) is used to cover at least a portion of the first wire 3571 and/or second wire 3581 to hold the first wire 3571 and/or second wire 3581 against the proximal hub 3545 and/or the catheter shaft 3503. In some examples, at least a portion of the proximal hub 3545 and/or the catheter shaft 3503 may include a groove 3547 for the first wire 3571 and/or second wire 3581 to be positioned in to secure the first wire 3571 and/or second wire 3581 in place. For example, the proximal hub 3545 may include a recessed channel 3549 where the first wire 3571 may be placed within to secure the location of the first wire 3571.

At least a portion of the proximal sensor transducer 3572 and/or at least a portion of the proximal sensor housing 3540 may be situated circumferentially between two of the strut feet. In the example shown in FIG. 35D, the proximal sensor transducer 3572 and the proximal sensor housing 3540 are positioned between the feet of a first strut 3551a and a second strut 3551b. This arrangement can allow for space efficiency and allow the sensor tranducer 3572 to be positioned within the outflow region of the blood pump.

The proximal sensor housing 3540 may be configured to position a pressure sensitive face 3573 of the proximal sensor transducer 3572 in a non-orthogonal orientation with respect to a long axis of the blood pump 3500. This arrangement may allow the pressure sensitive face 3573 to measure static pressure within the outflow region of the blood pump 3500 while minimizing or decreasing a dynamic pressure component of the measurement. In some examples, the pressure sensitive face 3573 is positioned parallel with respect to the long axis of the blood pump 3500.

Although the example of FIG. 35D shows the proximal sensor transducer 3572 on the proximal hub 3502, the proximal sensor transducer 3572 may be in alternative locations within the outflow region of the blood pump 3500. For example, the sensor transducer 3572 may be coupled to one of the struts 3551a/3551b or coupled to the blood conduit 3501.

The second wire 3581 may travel distally along one of the proximal struts, such as strut 3551b shown in FIG. 35D. The second wire 3581 may continue to travel distally along the blood conduit 3501 to the distal sensor transducer. The second wire 3581 may be sufficiently flexible to move with the strut 3551b and the blood conduit 3501 as they move radially outward (expand) and radially inward (collapse). In the example shown, the second wire 3581 travels along an outer surface of strut 3551b and an outer surface of the blood conduit 3501. However, the second wire 3581 may travel along any surface of the strut 3551b and the blood conduit 3501, such as inner surfaces of the strut 3551b and the blood conduit 3501 and/or along side edges of the strut 3551b. In some cases, the second wire 3581 may wind around at least a portion of the strut 3551b and/or the blood conduit 3501.

The second wire 3581 may be coupled to the strut 3551b and the blood conduit 3501 using any of a number of ways. For example, the second wire 3581 may be bonded (e.g., using adhesive, welding and/or molding) to the strut 3551b and the blood conduit 3501. In some cases, the second wire 3581 is bonded in selected locations such that the second wire 3581 has sufficient slack as the strut 3551b and the blood conduit 3501 transition between collaped and expanded states. In some examples, the strut 3551b and/or the blood conduit 3501 include one or more openings (e.g., gromets) for the second wire 3581 to run through and that allow the second wire 3581 to have sufficient slack as the strut 3551b and the blood conduit 3501 expand and collapse.

FIG. 35E illustrates a closeup view of the distal region 3562 (e.g., inflow region) of the catheter blood pump 3500. The distal region 3560 includes a distal hub 3522 that the distal ends 3523 of the struts 3553 are coupled to. The distal hub 3522 may refer to region of the catheter blood pump 3500 that is axially distally located with respect to the blood conduit 3501. The distal hub 3522 may have an outer surface that is radially inward with respect to the blood conduit 3501 when in an expanded state. In some cases, the distal hub 3522 houses a distal bearing assembly that may include one or more bearings (e.g., 1, 2, 3, 4, 5, etc.) associated with rotational movement of the drive cable 3512 (FIG. 35A). In some cases, a tapered bearing cap 3593 covers a portion of a proximal side of the distal hub 3522.

The distal hub 3522 may include a distal sensor housing 3542, which may be configured to secure a sensor transducer (e.g., pressure transducer) therein. The distal sensor housing 3542 may axially overlap with and/or be radially exterior to the bearing assembly within the distal hub 3522. The distal sensor housing 3542 may include a recess that is shaped and sized to accommodate the sensor therein. The sensor transducer may be mechanically fixed in distal sensor housing 3542 any of a number ways, such as described above with respect to the proximal sensor housing 3540. The distal sensor housing 3542 may include one or more openings (e.g., proximal opening 3555 and distal opening 3556) for one or more wires to extend out of the distal sensor housing 3542.

Like the proximal sleeve 3545, the distal sleeve 3595 may cover the distal strut feet 3523 and provide a radially inward force to retain/secure the distal struts 3553 to the distal hub 3522. In some cases, the distal sleeve 3595 may include a cutout 3568 to accommodate the distal sensor housing 3542. Like the proximal sleeve 3545, the distal sleeve 3595 may be mechanically coupled to the distal hub 3522 or may be free-floating with respect to the proximal hub 3502.

FIG. 35F illustrates a closeup view of the distal region 3562 of the catheter blood pump 3500 including an exemplary distal sensor transducer 3582 housed within the distal sensor housing 3542. In FIG. 35F, the distal sleeve 3595 is removed to provide a view of the underlying portion of the distal hub 3522. At least a portion of the distal sensor transducer 3582 and/or at least a portion of the distal sensor housing 3542 may be positioned between two of the distal struts 3553. In the example shown, the distal sensor transducer 3582 and the distal sensor housing 3542 are positioned between a first distal strut 3553a and a second distal strut 3553b. The distal sensor housing 3542 may be configured to position the pressure sensitive face of the distal sensor transducer 3582 in a non-orthogonal orientation with respect to a long axis of the blood pump 3500. This arrangement may allow the pressure sensitive face of the distal sensor transducer 3582 to measure static pressure within the inflow region of the blood pump 3500 while minimizing or decreasing a dynamic pressure component of the pressure measurement. In some examples, the pressure sensitive face of the distal sensor transducer 3582 is positioned parallel with respect to the long axis of the blood pump 3500.

The second wire 3581, which is attached to the distal sensor transducer 3582, may be arranged to exit the distal opening 3556 of the distal sensor housing 3542 and traverse within a recessed channel 3597 of the distal hub 3522. The channel 3597 may retain the second wire 3581 in a curved configuration that extends longitudinally and transversely with respect to the distal hub 3522. For example, the second wire 3581 may exit the distal opening 3556 and traverse in the distal direction, extend for a distance circumferentially around at least a portion of the distal hub 3522, traverse in the proximal direction along a third distal strut 3553c and the blood conduit 3501 toward the catheter shaft. This arrangment may allow for minimal bending/crimping of the second wire 3581.

In some variations, the channel 3597 may be configured to position the second wire 3571b to traverse a different strut, such as one of the first distal strut 3553a, the second distal strut 3553b or a fourth distal strut 3553d. One of the advantages of having the second wire 3581 traverse past at least one strut (e.g., second distal strut 3553b as shown in FIG. 35F) is that the longer circumferential pathway may reduce the extent of bending of the second wire 3581.

The proximal struts (e.g., 3551 in FIGS. 35A, 35B and 35D) and the distal struts (e.g., 3553 in FIGS. 35A, 35E and 35F) may include features that secure the struts radially with respect to respective proximal and distal hubs 3502/3522. For instance, in the example of FIG. 35F, the feet 3523 of the distal struts 3553 have t-shaped features with extensions that traverse circumferentially within a strut-retaining circumferential groove 3588 of the distal hub 3522. The feet of the proximal struts 3551 may include similar t-shaped features that traverse a corresponding strut-retaining annular groove in the proximal hub 3502.

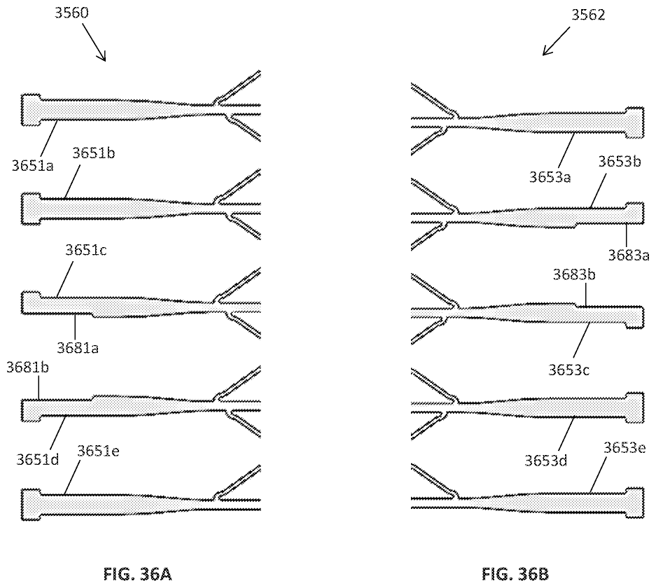

As described above, the blood conduit may have any number of struts. FIGS. 36A and 36B illustrate partial flattened views of an exemplary scaffold that may be used as part of any of the blood conduits described herein. FIG. 36A shows a proximal end 3560 of the scaffold, and FIG. 36B shows a distal end 3562 of the scaffold. The proximal end 3560 includes five struts 3651a, 3651b, 3651c, 3651d, 3651e, which may be coupled to a proximal hub (e.g., 3502). The distal end 3562 includes five struts 3653a, 3653b, 3653c, 3653d, 3653e, which may be coupled to a distal hub (e.g., 3522). As shown, each of the struts 3651/3653 may have t-shaped feet, which as described above, may help secure the scaffold to the proximal and distal hubs. In this example, two adjacent proximal struts 3651c and 3651d include cutouts 3681a and 3681b for accommodating a proximal sensor housing (e.g., 3540). Likewise, two adjacent distal struts 3653b and 3653c include cutouts 3683a and 3683b for accommodating a distal sensor housing (e.g., 3542). Such cutouts 3681a, 3681b, 3683a, 3683b may be used if circumferential space around the proximal and distal hubs is limited, for example, due to the number of struts.

Embodiments describe above include examples where the distal and/or proximal sensors are disposed between or adjacent to struts of the blood pump. In other embodiments, the distal and/or proximal sensors can be disposed distally to the struts, proximally to the struts, or alternatively, on or near a hub of the blood pump.

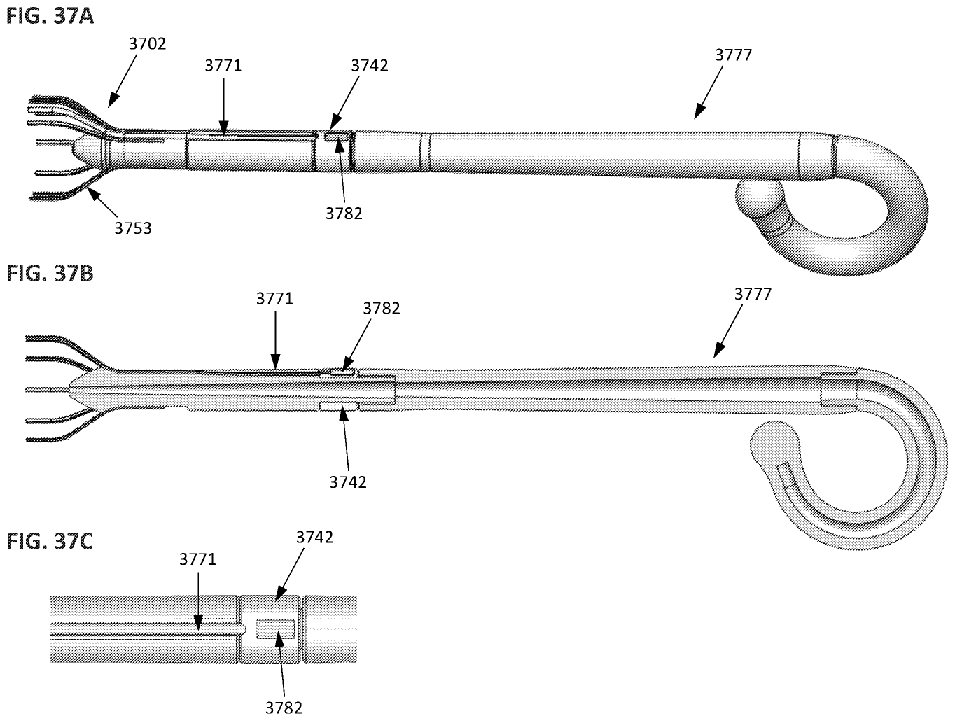

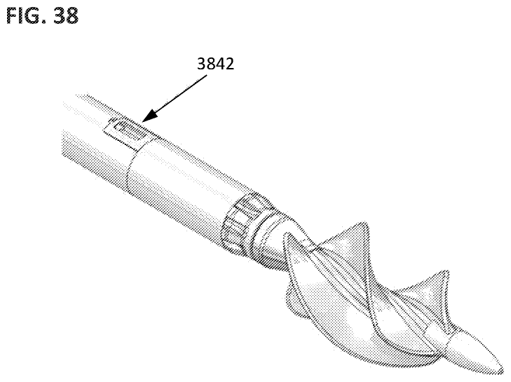

FIGS. 37A-37C illustrate another embodiment of a distal region of a catheter blood pump including an exemplary distal sensor transducer 3782 housed within a distal sensor housing 3742. In FIGS. 37A-37C, the distal sensor housing is positioned distally from the distal hub 3702 along a central portion of atraumatic distal tip 3777. In this embodiment, the distal sensor housing and distal sensor transducer are positioned a distance away from the hub 3702 so as to move the transducer away from areas or regions of potentially turbulent flow as the blood enters the blood pump conduit past struts 3753.

The distal sensor housing 3742 may be configured to position the pressure sensitive face of the distal sensor transducer 3782 in a non-orthogonal orientation with respect to a long axis of the blood pump. This arrangement may allow the pressure sensitive face of the distal sensor transducer 3782 to measure static pressure within the inflow region of the blood pump while minimizing or decreasing a dynamic pressure component of the pressure measurement. In some examples, the pressure sensitive face of the distal sensor transducer 3782 is positioned parallel with respect to the long axis of the blood pump.

The distal sensor housing 3742 may comprise an annular ring configured to be mounted to or mounted around the atraumatic distal tip 3777. In some embodiments, referring to the cutaway view of FIG. 37B, the atraumatic distal tip 3777 may have a section of reduced diameter or alternatively a cutout corresponding to the size of the distal sensor housing, so that when the distal sensor housing is mounted on the atraumatic distal tip it sits flush with the other surfaces of the atraumatic distal tip.

Referring still to FIGS. 37A-37C, the distal sensor transducer may be electrically coupled to a wire 3771, which can be configured to pass through the blood pump as previously described herein, including running along a portion of one or more struts 3753. In this embodiment, the atraumatic distal tip 3777 may include a cutout, lumen, or path configured to carry the wire 3771, so that the wire remains flush with the exterior surface of the atraumatic distal tip 3777 dista to the hub 3702 of the blood pump. FIG. 37C shows a view of the wire 3771 leading along the cutout in the distal tip up to the housing 3742 and sensor transducer 3782.

While the embodiment of FIGS. 37A-37C illustrates the distal region of the blood pump, including the distal tip and the distal hub, it should be understood that in another embodiment the same concepts can be applied to the proximal region of the blood pump. In this embodiment, the proximal sensor transducer can be positioned proximally from the proximal hub (instead of being positioned between the struts as shown and described above in reference to FIG. 35D).

1. A catheter blood pump, comprising:
a blood conduit defining a lumen and having a first end and a second end, wherein the first end is at an inflow region of the blood conduit and the second end is at an outflow region of the blood conduit;
an impeller positioned at least partially within the lumen of the blood conduit and configured to move blood from the inflow region toward the outflow region;
a plurality of struts extending from the first end or the second end of the blood conduit; and
a pressure transducer positioned between two adjacent struts in the inflow region or the outflow region of the blood conduit.

2. The catheter blood pump of claim 1, wherein the pressure sensor transducer has a pressure sensitive face facing outward relative to a long axis of catheter blood pump.

3. The catheter blood pump of claim 1, wherein the pressure transducer has a pressure sensitive face oriented non-orthogonally relative to a long axis of the catheter pump.

4. The catheter blood pump of claim 1, further comprising a pressure sensor housing in which the pressure transducer is securely disposed.

5. The catheter blood pump of claim 4, wherein the pressure sensor housing includes a proximal opening sized and configured to receive a pressure sensor wire coupled to the pressure transducer.

6. The catheter blood pump of claim 1, further comprising a pressure sensor wire extending proximally from the pressure transducer.

7. The catheter blood pump of claim 1, wherein the outflow region is at a proximal end of the blood conduit, wherein the plurality of struts extend in a proximal direction relative to the blood conduit.

8. The catheter blood pump of claim 7, wherein proximal ends of the plurality of struts are coupled to a hub of the catheter blood pump.

9. The catheter blood pump of claim 1, wherein the inflow region is at a distal end of the blood conduit, wherein the plurality of struts extend in a distal direction relative to the blood conduit.

10. The catheter blood pump of claim 9, wherein distal ends of the plurality of struts are coupled to a central hub of the catheter blood pump.

11. The catheter blood pump of claim 1, wherein the catheter blood pump includes a proximal pressure transducer in the outflow region and a distal pressure transducer in the inflow region.

12. The catheter blood pump of claim 11, further comprising a first wire electrically coupled to the proximal pressure transducer and a second wire electrically coupled to the distal pressure transducer.

13. The catheter blood pump of claim 12, wherein the first wire and the second wire extend together at a junction region, wherein the junction region is proximally located with respect to the blood conduit.

14. The catheter blood pump of claim 13, wherein the junction region is proximally located with respect to the proximal pressure sensor.

15. The catheter blood pump of claim 1, wherein the plurality of struts are expandable and collapsible.

16. The catheter blood pump of claim 1, wherein the blood conduit is expandable and collapsible.

17. The catheter blood pump of claim 1, wherein the impeller is expandable and collapsible.

18. The catheter blood pump of claim 1, further comprising a second impeller positioned at least partially within the blood conduit.

19. The catheter blood pump of claim 1, wherein the inflow region includes a distal end of the blood conduit and the outflow region includes a proximal end of the blood conduit.

20. The catheter blood pump of claim 1, wherein the pressure transducer is positioned circumferentially between the two adjacent struts of the plurality of struts, optionally circumferentially between feet (or axially extending regions) of the two adjacent struts.

21-83. (canceled)
